(19) United States
(12) Patent Application Publication
(10) Pub. No.: US 2026/0290499 A1
(43) Pub. Date: Sep. 24, 2026

(54) GENERATING BINDING FREE ENERGY UTILIZING INTELLIGENT RESTRAINTS AND LAMBDA STEPS FOR MOLECULAR DYNAMICS MODELS

(71) Applicant: Recursion Pharmaceuticals, Inc., Salt Lake City, UT (US)

(72) Inventors: Benjamin Philip COSSINS, Pewsey (GB); Gerhard KOENIG, London (GB); Zhiyi WU, Durham (GB)

(21) Appl. No.: 19/571,048

(22) Filed: Mar. 18, 2026

Related U.S. Application Data
(60) Provisional application No. 63/773,745, filed on Mar. 18, 2025.

(51) Int. Cl. G16B 15/30; G16B 5/30

(52) U.S. Cl. CPC G16B 15/30; G16B 5/30

(57) ABSTRACT

The present disclosure relates to systems, non-transitory computer-readable media, and methods for generating positional restraints and configuring lambda windows for execution within molecular dynamics simulation models. In particular, in one or more embodiments, the disclosed systems select restraint atoms based on geometry-conditioned loss metrics and apply positional restraints during staged lambda windows of an alchemical transformation to compute binding free energies between ligands and proteins. The disclosed systems generate candidate atoms, filter the candidate atoms based on angular criteria, and select restraint atom combinations using a loss function that penalizes angular variability and collinear geometries. In addition, in some embodiments, the disclosed systems configure and optimize lambda windows for annihilating nonbonded interactions using statistical free energy estimation techniques and variance-based selection. By coordinating restraint selection with lambda window scheduling, the disclosed systems improve stability and statistical reliability of molecular dynamics free energy calculations.

### CROSS-REFERENCE TO RELATED APPLICATIONS

This application claims priority to and the benefit of U.S. Provisional Patent Application No. 63/773,745, filed on Mar. 18, 2025, which is incorporated herein by reference in its entirety.

### BACKGROUND

Recent years have seen significant improvements in using molecular simulations to determine protein-ligand binding affinity for use in small molecule drug discovery. For example, conventional systems can quantify the binding affinity that can be used to assess and compare the strength of interaction between candidate small molecule ligands and a target protein in terms of a binding free energy. Conventional systems use molecular dynamics models to compute the binding free energy by performing various thermodynamic cycle calculations.

### SUMMARY

Embodiments of the present disclosure provide benefits and/or solve one or more of the foregoing or other problems in the art with systems, non-transitory computer-readable media, and methods for generating binding free energy calculations utilizing intelligent restraints and lambda steps in molecular dynamics simulations. For example, in some embodiments, the disclosed systems generate positional restraints between a ligand and a protein by selecting restraint atoms based on a loss function that penalizes ligand-protein geometric configurations that compromise stability during alchemical simulation. In one or more embodiments, the disclosed systems apply the restraints (utilizing an improved angular stabilization potential approach) during a series of lambda windows for generating a binding free energy using a molecular dynamics model. For instance, in some cases, the disclosed systems separately apply dihedral restraints and angle restraints in conjunction with annihilating various non-bonded interactions between the ligand and the protein across the lambda windows of the molecular dynamics model. Further, in one or more embodiments, the disclosed systems select a set of lambda windows to optimize restraints and/or annihilation of a nonbonded interaction. Additional features and advantages of one or more embodiments of the present disclosure are outlined in the description which follows and, in part, will be obvious from the description or may be learned by the practice of such example embodiments.

### BRIEF DESCRIPTION OF THE DRAWINGS

The detailed description provides one or more embodiments with additional specificity and detail through the use of the accompanying drawings, as briefly described below.

FIG. 1 illustrates an overview of a free energy optimization system generating and applying an optimized lambda schedule and applying restraints during a molecular dynamics simulation of an alchemical transformation to compute a binding free energy between a ligand and a protein in accordance with one or more embodiments.

FIG. 2 illustrates an example diagram of an overview of a free energy optimization system generating restraints that account for angular variability and collinear geometry and applying the restraints during lambda windows of a molecular dynamics model to generate a binding free energy in accordance with one or more embodiments.

FIG. 3 illustrates a schematic diagram of a free energy optimization system generating candidate atoms and generating a filtered set of candidate atoms from the candidate atoms in accordance with one or more embodiments.

FIG. 4 illustrates a schematic diagram of a free energy optimization system generating positional restraints by selecting restraint atoms from a filtered set of candidate atoms based on a loss function in accordance with one or more embodiments.

FIGS. 5A-5B illustrate schematic diagrams of a free energy optimization system applying restraints and annihilating nonbonded interactions during lambda windows of a molecular dynamics model in accordance with one or more embodiments.

FIG. 6 illustrates a schematic diagram of a free energy optimization system selecting an optimized set of lambda windows for annihilating a nonbonded interaction in accordance with one or more embodiments.

FIG. 7 illustrates a graph depicting the free energy optimization system generating binding free energies for known ligands with improved variability across replicate molecular dynamics cycles in comparison to conventional systems in accordance with one or more embodiments.

FIG. 8 illustrates graphs depicting reduced variability across lambda windows for binding free energy calculations in comparison to conventional systems in accordance with one or more embodiments.

FIG. 9 illustrates graphs depicting the free energy optimization system reducing variability between adjacent lambda windows during an alchemical transformation in comparison with conventional systems in accordance with one or more embodiments.

FIG. 10 illustrates a diagram of an environment in which a free energy optimization system can operate in accordance with one or more embodiments

FIG. 11 illustrates a flowchart of a series of acts for optimizing binding free energy calculations in molecular dynamics simulations in accordance with one or more embodiments.

FIG. 12 illustrates a block diagram of an example computing device for implementing one or more embodiments of the present disclosure.

### DETAILED DESCRIPTION

This disclosure describes one or more embodiments of a free energy optimization system that generates positional restraints and configures lambda windows for execution within molecular dynamics simulations that model thermodynamic cycles to compute binding free energies corresponding to ligand-protein affinity. In one or more embodiments, the free energy optimization system generates positional restraints between a ligand and a protein by selecting restraint atoms using a loss function that combines a measure of angular variability with an angular conditioning factor that penalizes configurations resulting in near-collinear geometries. The free energy optimization system can then apply the positional restraints during lambda windows of a molecular dynamics model (utilizing an improved angular stabilization potential approach) to generate a binding free energy. In some embodiments, the positional restraints include dihedral restraints and angle restraints that are applied during different sets of lambda windows while annihilating nonbonded interactions between the ligand and the protein. In addition, in some cases the free energy optimization system further selects lambda windows for annihilating a nonbonded interaction based on a statistical free energy estimation metric.

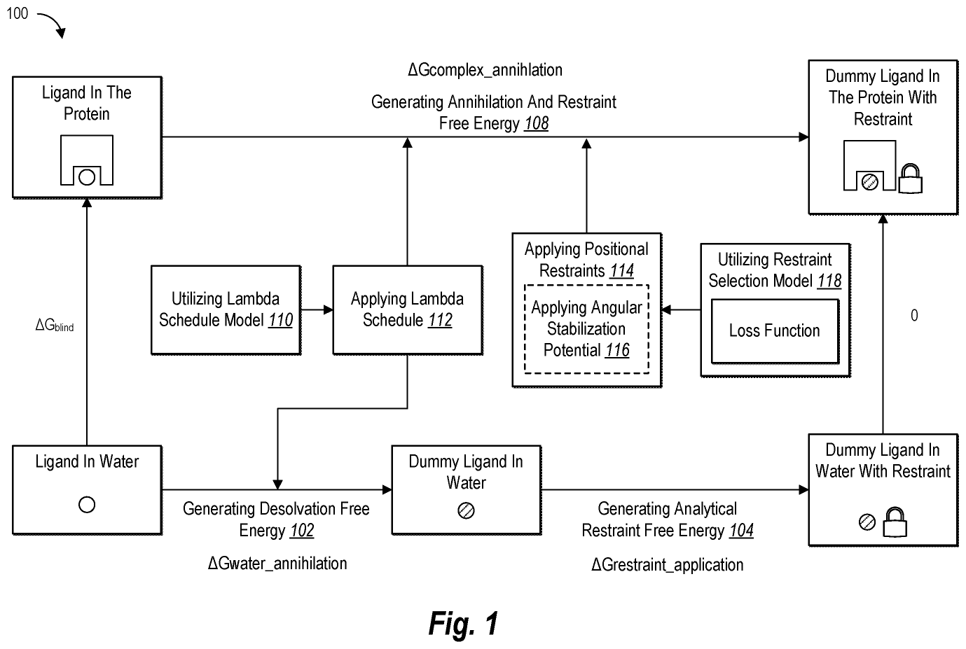

FIG. 1 illustrates an example overview of the free energy optimization system 100 generating a binding free energy using a thermodynamic cycle executed within a molecular dynamics model across optimized lambda windows and including application of positional restraints that utilize restraint atoms selected according to the previously mentioned loss function. In particular, FIG. 1 illustrates the free energy optimization system 100 utilizing the molecular dynamics model through a series of steps of the thermodynamic cycle to generate the binding free energy, including staged annihilation of nonbonded interactions and application of restraints that transform the ligand to a noninteracting state (e.g., a “dummy” ligand) while maintaining the ligand within a protein binding pocket. The free energy optimization system 100 generates the binding free energy by executing a plurality of transformations defined by the thermodynamic cycle and combining free energy values generated from the plurality of transformations. Additional details regarding the acts and processes introduced in relation to FIG. 1 are provided thereafter with reference to subsequent figures.

As shown in FIG. 1, the free energy optimization system 100 begins the thermodynamic cycle by performing a step 102 of generating a desolvation free energy (ΔG_{water_annihilation}) by modeling alchemically annihilating a ligand in water to transform the ligand into a “dummy” ligand. In particular, the free energy optimization system 100 generates the desolvation free energy by the alchemical annihilation of nonbonded interactions of the ligand in water across a series of lambda windows. For example, the free energy optimization system 100 simulates, within a molecular dynamics model, the alchemical annihilation of the ligand in an aqueous solvent environment, where the ligand is transformed from a fully interacting (e.g., physical) ligand into the dummy ligand and calculates the desolvation free energy corresponding to the transformation from the ligand to the dummy ligand.

As further shown, the free energy optimization system 100 performs a step 104 and generates an analytical restraint free energy (ΔG_{restraint_application}) by applying positional restraints between the ligand and the protein. More specifically, the free energy optimization system 100 applies positional restraints, for example angle restraints and dihedral restraints, that maintain a position between selected atoms of the ligand and corresponding reference atoms defining a protein-ligand pose. For example, the free energy optimization system 100 applies the positional restraints to the dummy ligand in water to generate a restrained dummy ligand state and generates the analytical restraint free energy associated with the applied restraints.

As shown, the free energy optimization system 100 performs a step 106 and models transfer of the restrained dummy ligand into the binding pocket of the protein. More particularly, the free energy optimization system 100 models moving the dummy ligand with the previously introduced positional restraints from the water to the protein to generate the dummy ligand in the protein with restraint. For instance, during step 106, the free energy optimization system 100 maintains the positional restraints while transferring the dummy ligand such that the dummy ligand remains non-interacting and nonbonded interactions between the dummy ligand and the protein are not active, consistent with the zero-free-energy transfer depicted in FIG. 1.

As further shown, in one or more embodiments, the free energy optimization system 100 performs a step 108 and generates an annihilation and restraint free energy (ΔG_{complex_annihilation}) while the ligand is in the binding pocket (or in the complex). In particular, the free energy optimization system 100 propagates a molecular dynamics model to generate the annihilation and restraint free energy by modeling deactivation of nonbonded interactions of the ligand in the protein to transform the ligand in the protein into a dummy ligand across a series of lambda windows, while applying the previously introduced positional restraints between selected ligand atoms and corresponding reference atoms.

In one or more embodiments, upon completing step 108 the free energy optimization system 100 can generate an overall binding free energy (ΔG_{bind}) between the ligand and the protein by computing the difference in free energy between the unbound ligand and the protein in water (e.g., the bottom portion of FIG. 1, step 102 and step 104) and the ligand in the binding pocket (e.g., the top portion of FIG. 1, step 108). For example, the free energy optimization system 100 generates the binding free energy (ΔG_{bind}) as follows: ΔG_{bind}=ΔG_{complex_annihlation}−(ΔG_{water_annihilation}+ΔG_{restraint_application}). In some cases, the free energy optimization system 100 generates a binding free energy by generating an absolute binding free energy. Moreover, the free energy optimization system 100 can also model restraint release free energy.

As previously mentioned, in one or more embodiments the free energy optimization system 100 annihilates nonbonded interactions across a series of lambda windows. In particular, the free energy optimization system 100 optimizes lambda windows for annihilating nonbonded interactions. For example, as illustrated, the free energy optimization system 100 performs a step 110 and utilizes a lambda scheduling model to generate lambda schedule comprising optimized lambda windows for annihilating nonbonded interactions and/or applying restraints. The free energy optimization system 100 can then perform a step 112 and apply the lambda schedule with the optimized lambda windows when annihilating nonbonded interactions, such as in step 102 and step 108 as shown in FIG. 1.

As used herein, “annihilating” or “alchemically annihilating” refers to reducing an interaction term toward zero, including fully eliminating or partially scaling the interaction via a coupling parameter. In certain implementations, annihilating includes scaling electrostatic and/or van der Waals interaction terms toward zero as a function of a coupling parameter, lambda. Thus, annihilating can include modifying non-bonded interaction potentials, adjusting coupling of non-bonded force field terms, scaling interaction parameters, progressively decoupling a molecular entity from its environment, or altering an interaction Hamiltonian as a function of a coupling parameter. In some embodiments, annihilating can include modifying intramolecular interactions and/or intermolecular interactions. Thus, annihilating non-bonded interactions does not require modifying or eliminating all non-bonded interactions but includes scaling or decoupling of certain non-bonded interactions (e.g., only intermolecular nonbonded interactions).

As also previously mentioned, the free energy optimization system 100 applies positional restraints to maintain position of a ligand and a protein when in the bound state while annihilating nonbonded interactions. In particular, the free energy optimization system 100 performs a step 118 and utilizes a restraint selection model to select restraint atoms for positional restraints between a ligand and a protein that avoid collinear geometric positions of 0° or 180° (where gradients to become numerically unstable). In one or more embodiments, as shown, the free energy optimization system 100 utilizes the restraint selection model that selects restraint atoms based on a loss function that penalizes angular variability and collinear geometry.

As also shown, the free energy optimization system 100 can perform a step 114 of applying the positional restraints to the protein-ligand configuration when annihilating nonbonded interactions, such as in step 108 as shown in FIG. 1. Moreover, while performing step 114, the free energy optimization system 100 can also perform a step 116 of applying an angular stabilization potential when applying certain positional restraints (e.g., angle restraints) to the protein-ligand configuration, which further reduces the likelihood that restraint angle values approach 0° or 180°.

Conventional systems that utilize molecular dynamics models suffer from a number of problems with regard to implementing computing devices when generating binding free energies. For instance, conventional systems inaccurately compute restraint forces in alchemical simulations when restrained angles approach singular boundary values. Specifically, conventional systems apply harmonic angle potentials that permit restrained angles to reach or approach 0° or 180° during molecular dynamics simulations. At those configurations, associated dihedral definitions become ill-conditioned, producing discontinuous or undefined gradients in the force calculation. This causes the molecular dynamics integrator to become numerically unstable, leading to simulation termination (“blow up”) in a measurable percentage of runs (e.g., approximately 5%). As a result, binding free energy values are not generated for those ligand-protein systems. Because of this absence of computed outputs for a subset of ligands, conventional systems create incomplete prediction datasets, reducing the accuracy and reliability of the overall computational screening, particularly in instances where the omitted ligands include high-affinity candidates.

Further, conventional systems are inflexible as they implement lambda scheduling using fixed, pre-defined scaling patterns and static interaction ordering that are not responsive to system-specific statistical behavior. Specifically, conventional systems distribute lambda windows according to hard-coded linear or semi-linear progressions and apply predetermined rules for scaling nonbonded interactions in a fixed sequence. These distributions of lambda windows and interaction schedules are protocol-dependent and remain constant across different ligand-protein systems. Because the scheduling logic is statically defined within the computational architecture, conventional systems execute the same lambda configuration regardless of differences in molecular topology, interaction strength, or statistical variance behavior. This fixed scheduling structure limits the ability of the computer system to reconfigure its internal execution parameters in response to system-specific computational characteristics, thereby constraining architectural flexibility of the simulation engine.

Moreover, conventional systems are inefficient as they excessively consume computational resources when repeatedly executing thermodynamic simulations with geometric configurations and static lambda window scheduling that result in unstable simulations and/or elevated estimator variance, thereby requiring repeat runs. Specifically, conventional systems execute fixed lambda distributions and interaction progressions that produce localized variance spikes and, in some cases, simulation termination events caused by unstable restraint geometries. When a simulation terminates prematurely or produces statistically unstable output, the processor cycles consumed during that execution do not yield a usable binding free energy result, and conventional systems must execute additional simulation runs to obtain stable estimates. These repeated executions by conventional systems increase total processor utilization, while also prolonging runtime for large-scale molecular simulation workflows.

The free energy optimization system provides a variety of technical advantages relative to conventional systems. For example, by preferentially selecting restraint geometries with a lower likelihood of approaching or reaching 0° or 180° and then applying an angular stabilization potential while applying positional restraints during the lambda windows, the free energy optimization system 100 improves computational accuracy relative to conventional systems. Specifically, the free energy optimization system 100 selects restraint atoms for positional restraints by using a loss function that penalizes large geometric fluctuations, long anchor distances, and angular values approaching threshold angular geometries (e.g., 0° or 180°), thereby preferentially selecting restraint configurations whose equilibrium angles lie within a stable mid-range. By reducing the probability that restrained angles drift toward threshold angular geometries during simulation and further applying an angular stabilization potential having a flat-bottom region within an operational range and a non-linearly increasing gradient near boundary values, the free energy optimization system 100 increases restorative force as restraints approach threshold angular geometries while avoiding introduction of excessively stiff force constants that would create numerically unstable vibrational modes. Together, the structured restraint selection and boundary-limiting angular stabilization potential eliminate undefined gradient conditions in the force calculation, prevent numerical divergence of the molecular dynamics integrator, and enable completion of simulations without threshold geometry-induced termination, thereby improving numerical stability, determinism, and completeness of generated binding free energy outputs relative to conventional systems.

In addition, the free energy optimization system 100 improves configurability and architectural flexibility of the computer-implemented free energy calculation workflow relative to conventional systems by deriving scheduling of lambda windows from computational feedback rather than fixed protocol definitions. Specifically, the free energy optimization system 100 generates lambda windows and modifies the order and rate at which nonbonded interactions are varied during the alchemical transformation based on measured statistical characteristics of a free energy estimator. For example, the free energy optimization system 100 evaluates estimator-derived error metrics across candidate lambda windows and selects a distribution of lambda windows that minimizes estimator variance for the molecular system under analysis. Because the lambda window placement and interaction progression are determined through computational evaluation of system-specific statistical behavior, the free energy optimization system 100 configures its internal execution parameters based on the particular ligand-protein system the free energy optimization system 100 is processing.

Moreover, the free energy optimization system 100 improves efficiency relative to conventional systems by reducing simulation termination events and minimizing estimator variance through optimized restraint selection and variance-driven lambda scheduling. By preventing threshold geometry-induced instability and by redistributing lambda windows to reduce statistical variance, the free energy optimization system 100 reduces the need for repeated simulation runs to achieve stable convergence. Because each execution of the molecular dynamics engine is more likely to complete successfully and produce statistically stable output, the free energy optimization system 100 decreases total processor cycles required per ligand and shortens cumulative runtime for multi-ligand simulation campaigns. This constitutes a direct improvement in computational resource utilization of the computer-implemented free energy calculation workflow. Indeed, in some implementations, the free energy optimization system 100 significantly reduced (and even eliminated) computer termination/crashes in executing molecular dynamic simulation models.

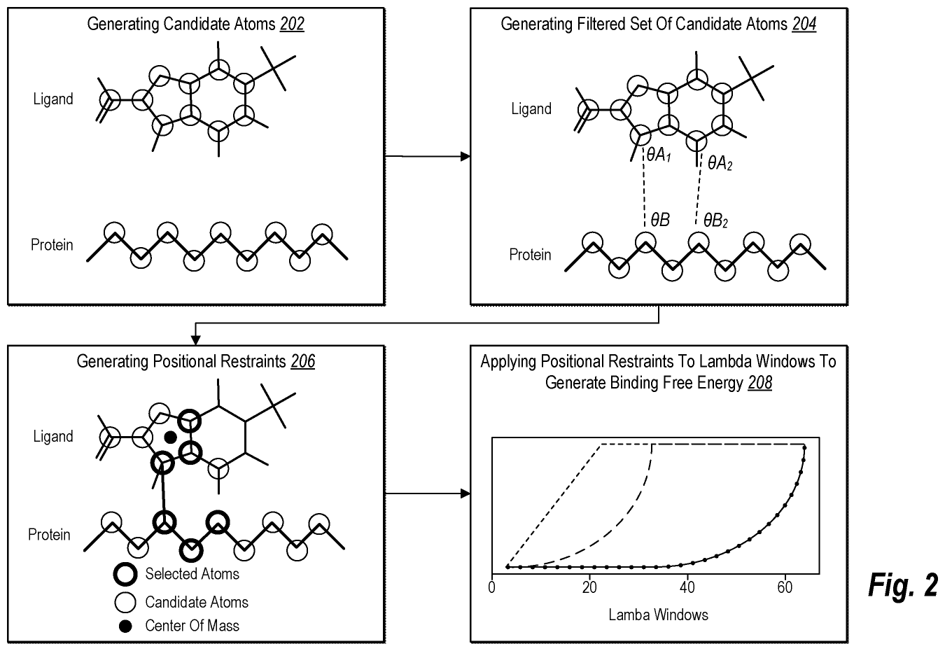

As previously mentioned, in one or more embodiments, the free energy optimization system 100 optimizes generating binding free energies by selecting restraint geometries and generating lambda windows that penalize near-collinear geometries. More specifically, the free energy optimization system 100 selects restraint atoms to generate positional restraints by using a loss function that accounts for angular variability and collinear variability and applies the restraints according to optimized lambda windows generated by the free energy optimization system 100. FIG. 2 illustrates an example diagram of an overview of a free energy optimization system generating restraints that account for angular variability and collinear geometry and applying the restraints during lambda windows of a molecular dynamics model to generate a binding free energy in accordance with one or more embodiments.

As illustrated in FIG. 2, the free energy optimization system 100 performs a step 202 of generating candidate atoms. More specifically, the free energy optimization system 100 identifies one or more ligand atoms and one or more protein atoms that are suitable for generating positional restraints within a molecular dynamics model. For example, the free energy optimization system 100 may generate an initial set of candidate atoms for further geometric evaluation by selecting non-terminal atoms connected to at least two heavy atoms.

As shown, the free energy optimization system 100 performs a step 204 of generating a filtered set of candidate atoms. In particular, the free energy optimization system 100 evaluates geometric relationships between the candidate ligand atoms and candidate protein atoms to determine whether the candidate atoms satisfy one or more geometric criteria. Based on the evaluation, the free energy optimization system 100 excludes candidate atoms associated with geometries that are unsuitable for stable positional restraint generation, thereby producing a filtered set of candidate atoms for subsequent restraint selection. Additional details regarding the free energy optimization system 100 generating candidate atoms and generating a filtered set of candidate atoms are provided below with respect to FIG. 2.

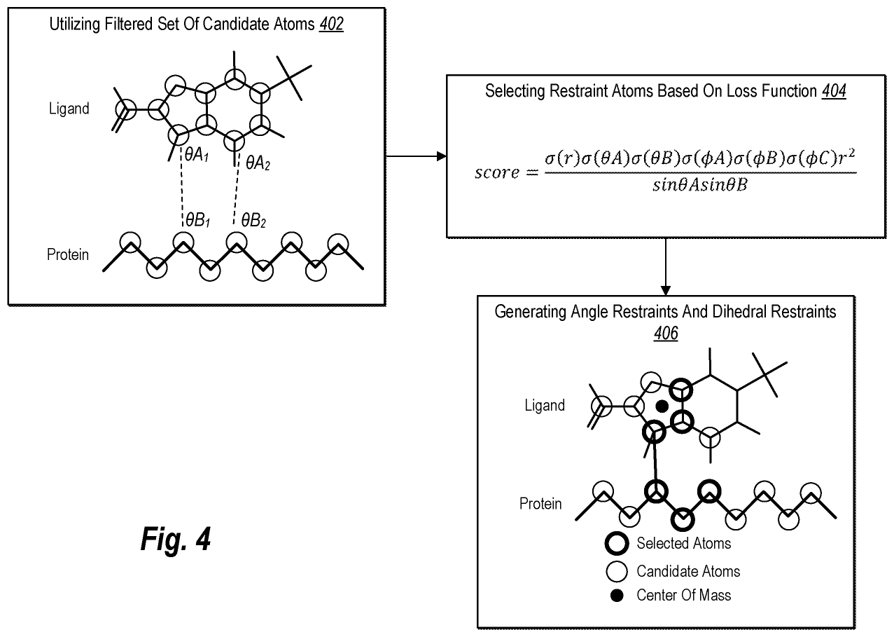

In addition, as shown, the free energy optimization system 100 performs a step 206 of generating positional restraints. Specifically, the free energy optimization system 100 generates positional restraints by simulating restraints by selecting restraint atoms from the filtered set of candidate atoms and generating positional restraints (e.g., angle restraints and dihedral restraints) using the selected restraint atoms that maintain a position of a ligand bound to a protein with annihilating nonbonded interactions. In some instances, the free energy optimization system 100 selects restraint atoms based on a loss function that combines a measure of angular variability that penalizes angular variability with an angular conditioning factor that penalizes collinear geometry. Additional details regarding the free energy optimization system 100 selecting restraint atoms from the filtered set of candidate atoms based on a loss function are provided below with respect to FIG. 4.

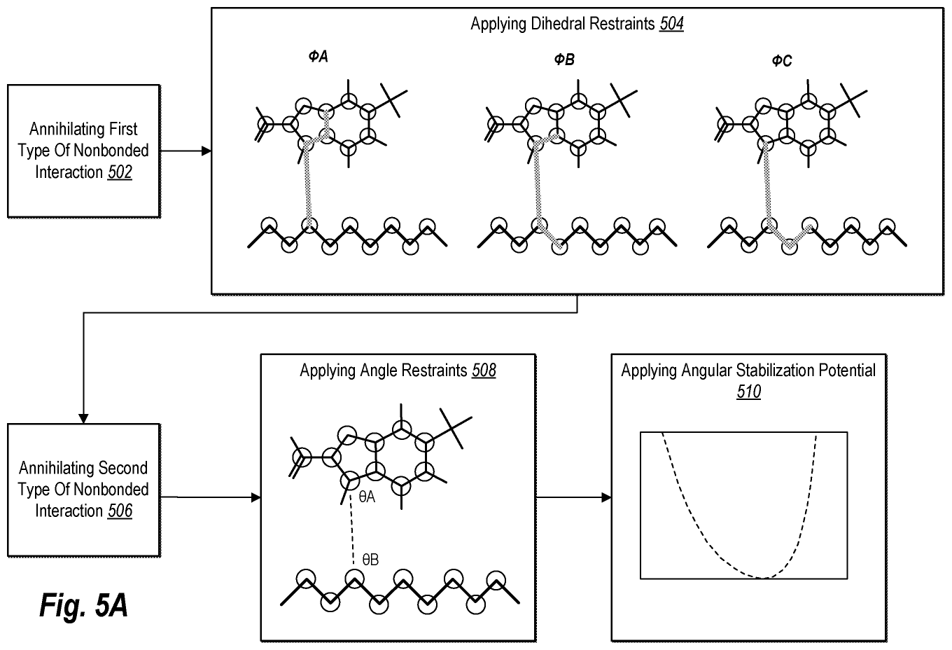

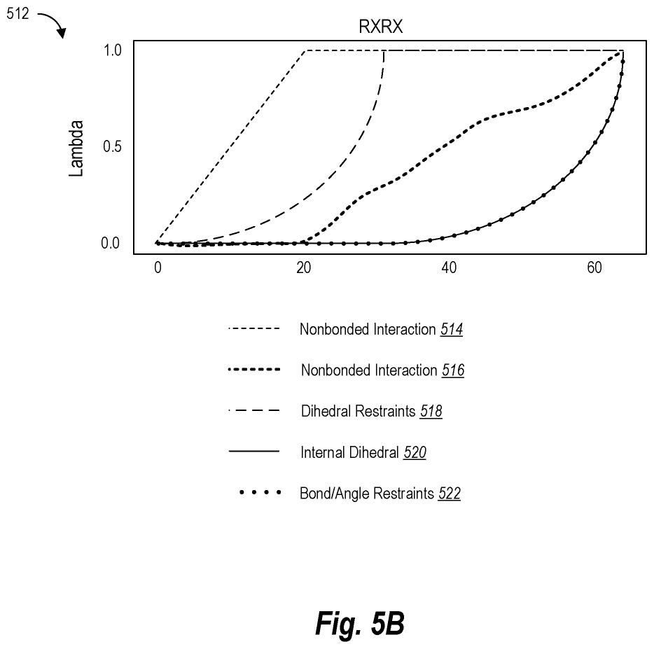

As shown, the free energy optimization system 100 can perform a step 208 of applying positional restraints to lambda windows to generate binding free energy. More specifically, in some embodiments, the free energy optimization system 100 generates a plurality of lambda windows defining intermediate stages of an alchemical transformation within a molecular dynamics model. The free energy optimization system 100 can then apply the positional restraints during execution of the lambda windows while also annihilating nonbonded interactions between a ligand and a protein in accordance with lambda parameters. The free energy optimization system 100 can then utilize simulation data from across the lambda windows to compute a binding free energy for the ligand-protein system. Additional details regarding the free energy optimization system 100 generating and applying positional restraints and annihilating nonbonded interactions across lambda window are provided below with respect to FIGS. 5A-5B.

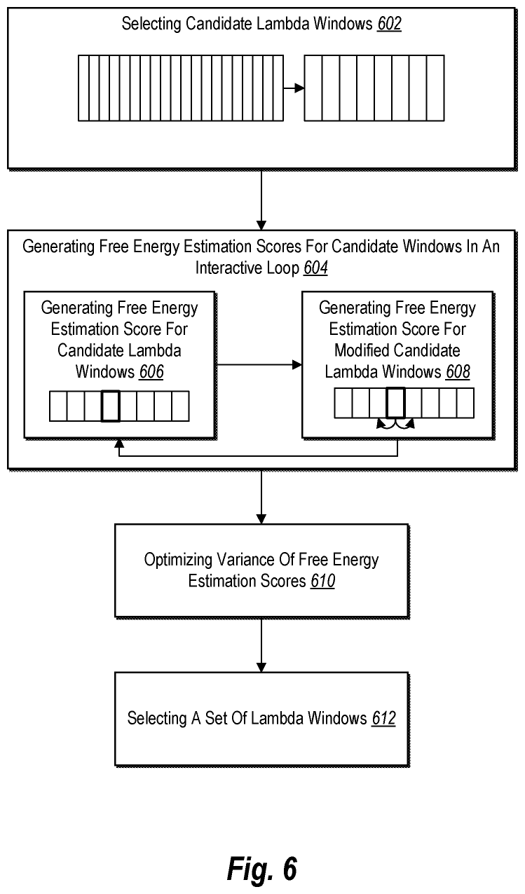

As just mentioned, in one or more embodiments, the free energy optimization system 100 generates lambda windows that define intermediate stages for annihilating nonbonded interactions and applying positional restraints. More specifically, the free energy optimization system 100 can evaluate candidate lambda window schedules to determine an arrangement of lambda windows that promotes stable thermodynamic transformations and reliable free energy estimation. For instance, the free energy optimization system 100 analyzes simulation metrics associated with candidate lambda window schedules and selects a set of lambda windows based on statistical free energy estimation criteria. Additional details regarding the free energy optimization system 100 selecting a set of lambda windows are provided below with respect to FIG. 6.

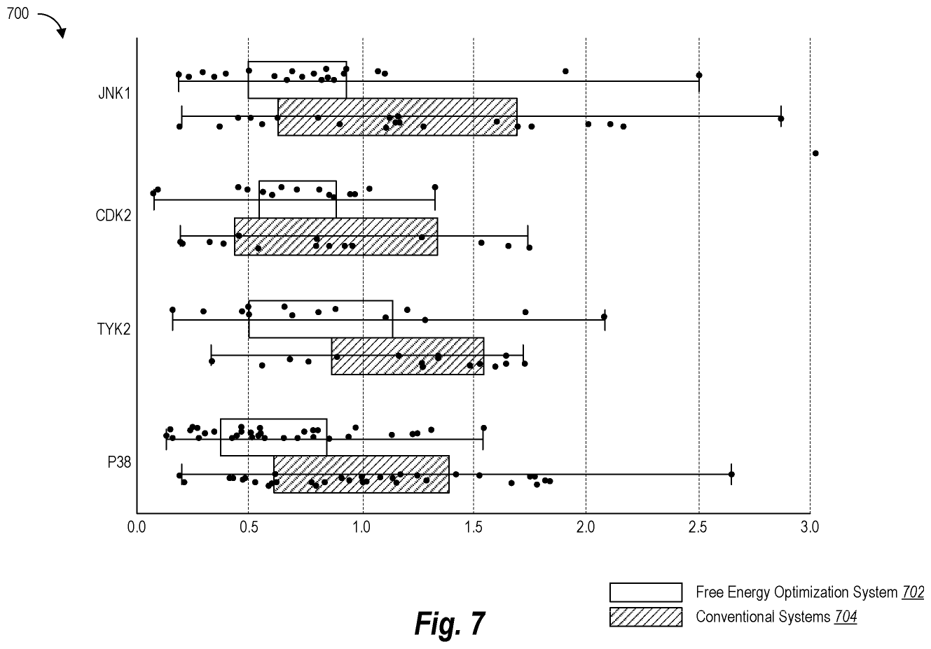

As previously mentioned, the free energy optimization system 100 improves accuracy, flexibility, and efficiency over conventional systems. In particular, the free energy optimization system 100 improves stability, convergence, and statistical reliability of binding free energy calculations through geometry-conditioned restraint selection and optimized lambda window configurations that result in enhanced agreement between computed and reference binding free energies, reduced variability across simulation runs, and more consistent free energy estimation. Additional details and example graphs regarding improvements of the free energy optimization system 100 are provided in FIGS. 7-9 and the accompanying descriptions below.

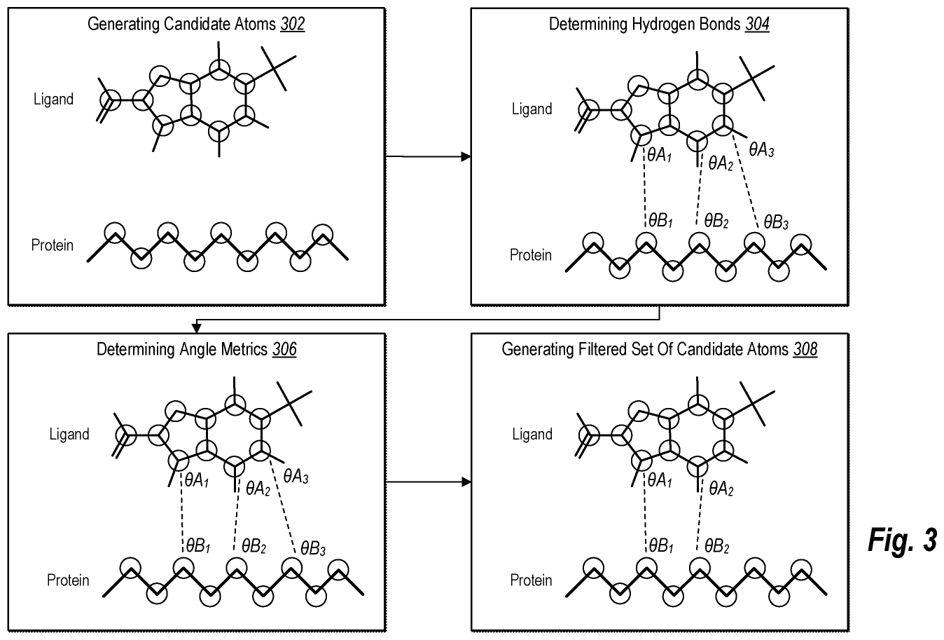

As previously mentioned, the free energy optimization system 100 generates candidate atoms and excludes candidate atoms to generate a filtered set of candidate atoms. In particular, the free energy optimization system 100 generates the filtered set of candidate atoms by excluding candidate atoms that do not satisfy an angle metric threshold. FIG. 3 illustrates a schematic diagram of a free energy optimization system generating candidate atoms and generating a filtered set of candidate atoms from the candidate atoms in accordance with one or more embodiments.

As illustrated in FIG. 3, the free energy optimization system 100 performs a step 302 of generating candidate atoms. As used herein, the term “candidate atoms” refers to one or more atoms that are eligible for selection as part of a restraint in a molecular dynamics simulation. In particular, the term “candidate ligand atoms” can include ligand atoms that satisfy one or more structural or chemical criteria associated with geometric stability, bonding configuration, or participation in intermolecular interactions. To illustrate, candidate atoms can include non-hydrogen ligand atoms that are connected to at least two heavy atoms, such as carbon, nitrogen, oxygen, or sulfur atoms. In addition, the term “candidate protein atoms” can include protein atoms that are eligible for participation in defining a restraint with a ligand, including protein atoms associated with intermolecular interactions between the ligand and the protein, backbone atoms, side-chain atoms, or other atoms selected based on structural, geometric, or interaction-based criteria.

As previously mentioned, in one or more embodiments, the free energy optimization system 100 generates candidate atoms by selecting atoms from a ligand as an initial set of atoms for downstream evaluation in determining restraint atom combinations. In some instances, the free energy optimization system 100 selects non-hydrogen atoms of a ligand that are connected to at least two heavy atoms as candidate atoms (e.g., candidate ligand atoms) for generating positional restraints. For example, for a six-atom ligand-protein positional restraint configuration that includes 3 atoms from a ligand and 3 atoms from a protein, the candidate atoms include options for downstream selection of 3 atoms for the ligand portion of positional restraints between the ligand and the protein.

As further illustrated in FIG. 3, the free energy optimization system 100 performs a step 304 of determining hydrogen bonds. Specifically, the free energy optimization system 100 enumerates hydrogen bonds between the ligand and the protein based on simulated binding interactions and mapping the hydrogen bonds. For example, the free energy optimization system 100 enumerates hydrogen bonds observed during simulation and maps hydrogen bonds between the ligand and the protein by mapping the ligand part of the hydrogen bond to the candidate atoms generated in step 302 and the protein part of the hydrogen bond to a C_{α}-carbon of the corresponding residue of the protein. In certain embodiments, the free energy optimization system 100 associates protein residues participating in the hydrogen bonds with corresponding protein atoms for subsequent restraint atom evaluation.

Further, in one or more embodiments, the free energy optimization system 100 selects a subset of hydrogen bonds from the determined hydrogen bonds. For instance, the free energy optimization system 100 selects hydrogen bonds based on an occupancy criterion, such as when hydrogen bonds have an occupancy that satisfies a threshold occupancy (e.g., greater than 50%) during the simulation. Further, in some cases, the free energy optimization system 100 can consider various combinations of candidate atoms associated with the selected hydrogen bonds. For example, the free energy optimization system 100 can determine hydrogen bonds that correspond to candidate six-atom combinations, including three candidate ligand atoms from the ligand (as determined in step 302) and three candidate protein atoms from a backbone of the protein (e.g., Ca, N, C) corresponding to residues participating in the selected hydrogen bonds.

As further shown, the free energy optimization system 100 can perform a step 306 and determine angle metrics. As used herein, the term “angle metrics” refers to one or more quantitative measures that characterize an angular relationship between atoms of a ligand and atoms of a protein. In some embodiments, an angle metric can correspond to a geometric angle formed by three atoms, such as an angle defined by two ligand atoms and one protein atom or vice versa. In certain implementations, angle metrics can include angles corresponding to θA and θB, or other angular values derived from positional coordinates of candidate atoms within a molecular dynamics model. Specifically, θA is defined by the angle between (1) a line between a first atom of the protein and a first atom of the ligand and (2) a line between the first atom of the ligand and a second (adjacent or connected) atom of the ligand. Similarly, θB is defined by the angle between (1) a line between a first atom of the ligand and the first atom of the protein and (2) a line between the first atom of the protein and a second (adjacent or connected) atom of the protein.

In one or more embodiments, the free energy optimization system 100 determines one or more angle metrics between candidate ligand atoms and candidate protein atoms based on the hydrogen bonds determined in step 304. More specifically, the free energy optimization system 100 generates angle metrics by computing values corresponding to geometric angle relationships formed by combinations of candidate ligand atoms and candidate protein atoms. For example, the free energy optimization system 100 may evaluate angles defined by selected ligand atoms and protein backbone atoms associated with residues participating in the identified hydrogen bonds. As an illustration, the free energy optimization system 100 can determine angle metrics by computing values for θA₁, θA₂, θA₃, θB₁, θB₂, and θB₃.

As shown, the free energy optimization system 100 can also perform a step 308 of generating a filtered set of candidate atoms. As used herein, the term “filtered set of candidate atoms” refers to a subset of candidate atoms that remains after applying one or more selection criteria to a plurality of candidate atoms. In some embodiments, the selection criteria can include geometric, spatial, energetic, statistical, or other quantitative constraints applied to relationships between atoms of a ligand and atoms of a protein. The filtered set of candidate atoms can therefore represent those candidate atoms that satisfy one or more predefined thresholds or conditions for further processing within a molecular dynamics model.

In some embodiments, the free energy optimization system 100 can generate the filtered set of candidate atoms by selecting a subset of candidate atoms from a plurality of candidate atoms based on angle metrics as determined in step 306 satisfying an angle metric threshold. As used herein, the term “angle metric threshold” refers to one or more predetermined criteria used to evaluate whether an angle metric satisfies a condition for further processing. In some embodiments, the angle metric threshold can include a minimum value, a maximum value, a range of values, or another comparative condition applied to one or more angle metrics. The angle metric threshold can be defined based on geometric, statistical, empirical, or model-based considerations within a molecular dynamics framework.

In some cases, the free energy optimization system 100 generates the filtered set of candidate atoms by selecting (or retaining) candidate atom combinations that satisfy an angle metric threshold and excludes candidate atom combinations that do not satisfy the angle metric threshold. The resulting subset of candidate atoms forms the filtered set of candidate atoms for subsequent positional restraint selection. In some cases, the free energy optimization system 100 selects (or retains) candidate atom combinations where angle metrics fall within an angle metric range, such as where OA and OB fall within a range of 45° and 135°.

In addition, the free energy optimization system 100 can further refine the filtered set of candidate atoms based on placement of candidate ligand atoms within the ligand. More specifically, the free energy optimization system 100 can select candidate atoms that are closest to the ligand's center of mass. For example, among remaining candidate atom combinations that satisfy the angle metric threshold, the free energy optimization system 100 can select combinations in which a candidate ligand atom has a minimum distance to a center of mass of the ligand relative to other candidate ligand atoms.

As previously mentioned, in one or more embodiments the free energy optimization system 100 generates positional restraints that maintain a position between a ligand and a protein. In particular, the free energy optimization system 100 evaluates combinations of filtered candidate atoms using a loss function to identify restraint atoms for forming the positional restraints. FIG. 4 illustrates a schematic diagram of a free energy optimization system generating positional restraints by selecting restraint atoms from a filtered set of candidate atoms based on a loss function in accordance with one or more embodiments.

As illustrated in FIG. 4, the free energy optimization system 100 performs a step 402 and utilizes a filtered set of candidate atoms (as generated in step 308 of FIG. 3) to perform a step 404 of selecting restraint atoms based on a loss function. As used herein, the term “restraint atoms” refers to one or more atoms selected for use in defining a positional restraint between a ligand and a protein in a molecular dynamics model. In some embodiments, restraint atoms can be selected from a filtered set of candidate atoms based on one or more selection criteria. For instance, restraint atoms can serve as reference points for defining geometric relationships, including distances, angles, or dihedral angles, within a molecular dynamics model. In some cases, restraint atoms include physical atoms, pseudo-atoms, virtual sites, centroids, center-of-mass representations, or other coordinate-defined reference points derived from one or more atoms of the ligand or the protein.

As mentioned, in one or more embodiments, the free energy optimization system 100 selects restraint atoms based on a loss function. In particular, the free energy optimization system 100 evaluates combinations of candidate atoms from the filtered set using the loss function to identify a subset of atoms for generating positional restraints between the ligand and the protein. In particular, the free energy optimization system 100 generates loss metric for restraint atom combinations of the filtered set of candidate atoms using a loss function that combines a measure of angular variability and an angular conditioning factor that penalizes collinear geometry and select the restraint atoms that give the lowest score. As used herein, the term “measure of angular variability” refers to one or more quantitative values that characterize variability of one or more angular parameters across a set of configurations or simulation states (e.g., standard deviation). Specifically, the term “measure of angular variability” can refer to quantitative values that characterize variability of angular parameters for positional restraints, such as angle restraints or dihedral restraints. In some embodiments, the measure of angular variability can reflect dispersion, fluctuation, or spread of angular values associated with positional restraints during a molecular dynamics simulation. Further, the measure of angular variability can be determined using one or more statistical or computational techniques and can be combined with additional terms (e.g., within a loss function).

As used herein, the term “angular conditioning factor” refers to a quantitative factor applied to adjust evaluation of restraint geometries based on one or more instantaneous angular configurations. In some embodiments, the angular conditioning factor increases a penalty as one or more angles approach collinear or otherwise geometrically unstable configurations. The angular conditioning factor can be implemented using one or more mathematical functions of angular values and can modify a loss function independently of measures of angular variability.

As mentioned, an angular conditioning factor can penalize collinear geometry. As used herein, the term “collinear geometry” refers to a geometric configuration in which three or more atoms are arranged substantially along a single straight line or axis. In some embodiments, collinear geometry can occur when an angle formed by three atoms approaches 0 degrees or 180 degrees or otherwise approaches a configuration in which the atoms are aligned. Such collinear geometries can introduce numerical instability, undefined or ill-conditioned mathematical behavior, or excessive forces within a molecular dynamics simulation, potentially leading to divergence or failure of the simulation.

As mentioned, the free energy optimization system 100 generates loss metrics based on a loss function that combines a measure of angular variability and an angular conditioning factor that penalizes collinear geometry. In particular, the free energy optimization system 100 can generate a loss metric based on one or more of the following: angle values between the ligand and protein, dihedral angle values between the ligand and the protein, distance between the ligand and the protein, measure of variability of angle values between the ligand and protein, measure of variability of dihedral angle values between the ligand and the protein, measure of variability of distance between the ligand and the protein, or angular conditioning factor. The free energy optimization system 100 can utilize a variety of combinations of these factors in determining the loss metrics. For example, the loss function can be based on angle values between the ligand and protein and one or more of the following dihedral angle values between the ligand and the protein, distance between the ligand and the protein, measure of variability of angle values between the ligand and protein, measure of variability of dihedral angle values between the ligand and the protein, measure of variability of distance between the ligand and the protein, or angular conditioning factor. Similarly, the loss function can be based on dihedral angle values between the ligand and the protein and one or more of the following: distance between the ligand and the protein, measure of variability of angle values between the ligand and protein, measure of variability of dihedral angle values between the ligand and the protein, measure of variability of distance between the ligand and the protein, or angular conditioning factor. Moreover, the loss function can be based on distance between the ligand and the protein and one or more of the following: angle values between the ligand and protein, dihedral angle values between the ligand and the protein, measure of variability of angle values between the ligand and protein, measure of variability of dihedral angle values between the ligand and the protein, measure of variability of distance between the ligand and the protein, or angular conditioning factor. In addition, the loss function can be based on any one of the measures of variability described herein in combination with one or more of the other factors described herein.

In one or more embodiments, the free energy optimization system 100 utilizes the following loss function:

$$loss metric = \frac{σ ( r ) σ ( θ A ) σ ( θ B ) σ ( ϕ A ) σ ( ϕ B ) σ ( ϕ C ) {r}^{2}}{sin θ A sin θ B}$$

where the loss metric is based on the standard deviation (σ) of the distance (r in nm), the angle values (θA and θB in radian), and the dihedral angle values (φA, φB and φC in radian). In particular, the measure of angular variability comprises at least the standard deviation values associated with one or more angular parameters of the restraint atom combinations, including standard deviation values of angle parameters (e.g., σ(θA), σ(θB)) and, in some embodiments, standard deviation values of dihedral angle parameters (e.g., σ(φA), σ(φB), σ(φC)). The angular conditioning factor that penalizes collinear geometry corresponds to a denominator term that depends on sin functions of one or more angle parameters (e.g., sin θA and sin θB), such that the loss metric increases as the angle parameters approach collinear configurations. In some embodiments, the loss function can further include a distance-based term (e.g., r² and σ(r)) that contributes to evaluation of restraint atom combinations.

As shown above, in some embodiments, the angular conditioning factor comprises a denominator term including a product of sin functions of one or more angle parameters, such that the loss metric is inversely proportional to sin (θA) and sin (θB). In such embodiments, the loss metric increases as θA or θB approaches values at which sin (θ) approaches zero, thereby penalizing near-collinear geometries through amplification of the loss metric. The free energy optimization system 100 selects restraint atoms corresponding to a minimum loss metric computed using the sine-based denominator formulation.

In some embodiments, the loss function can alternatively include an angular conditioning factor implemented as a multiplicative term that depends on (1−sin θA) and (1−sin θB), such that:

$$loss metric = σ ( r ) σ ( θ A ) σ ( θ B ) σ ( ϕ A ) σ ( ϕ B ) σ ( ϕ C ) {r}^{2} ( 1 - sin θ A ) ( 1 - sin θ B )$$

In such embodiments, the angular conditioning factor increases the loss metric as θA or θB approaches a collinear configuration. Both implementations operate to penalize geometries in which the angle parameters approach 0° or 180° during partial or full annihilation of nonbonded interactions. Indeed, either loss function, together with the previously described filtering steps (e.g., to generate the filtered set of candidate atoms) biases selection toward configurations in which θA and θB remain away from 0° or 180° during partial or full annihilation of the ligand's nonbonded interactions, and that the protein component corresponds to a structurally stable region.

As mentioned, the free energy optimization system 100 can select restraint atoms based on the loss metrics. In one or more embodiments, the free energy optimization system 100 generates a loss metric for each restraint atom combination of the filtered set of candidate atoms using one of the loss functions described above. In particular, the free energy optimization system 100 computes a respective loss metric value for multiple restraint atom combinations and compares the computed loss metric values to identify one or more combinations associated with lower loss metric values. For instance, the free energy optimization system 100 selects a restraint atom combination corresponding to a minimum or comparatively reduced loss metric value relative to other evaluated combinations.

As further illustrated in FIG. 4, the free energy optimization system 100 performs a step 406 of generating positional restraints. In particular, the free energy optimization system 100 generates the positional restraints between a ligand and a protein based on the restraint atoms selected in step 404. As used herein, the term “positional restraints” refers to constraints, potentials, or forces applied within a molecular dynamics model to regulate spatial relationships between one or more atoms of a ligand and one or more atoms of a protein. In some embodiments, positional restraints can regulate one or more geometric parameters, including distances, angles, dihedral angles, or combinations thereof. For example, the positional restraints can be implemented using one or more functions configured to influence relative positioning during simulation. Further, the positional restraints can be static or dynamic and can be selectively applied, modified, scaled, or activated during one or more stages of a molecular dynamics simulation, including at various different intermediate stages of simulation.

As mentioned, in one or more embodiments, the free energy optimization system 100 generates the positional restraints using the restraint atoms. In particular, the free energy optimization system 100 generates the restraints that specify geometric parameters associated with the selected restraint atoms for use within a molecular dynamics model. For example, the free energy optimization system 100 can generate one or more restraint definitions that include target geometric values and corresponding forces associated with distances, angles, dihedral angles, or combinations thereof between ligand atoms and protein atoms. In some embodiments, the generated positional restraints are configured to regulate relative spatial relationships between the ligand and the protein during execution of the molecular dynamics model.

In some embodiments, the free energy optimization system 100 generates positional restraints by generating one or more angle restraints and one or more dihedral restraints, or combinations thereof, based on the selected restraint atoms. For example, the free energy optimization system 100 can generate a set of positional restraints that includes one or more angle restraints defined by three atoms and one or more dihedral restraints defined by four atoms, where the atoms are selected from ligand atoms and protein atoms identified in the selected restraint atom combination (e.g., in step 404).

As used herein, the term “angle restraint” refers to a positional restraint configured to regulate or influence an angle formed by three atoms within a molecular dynamics model. In some embodiments, an angle restraint applies a potential, force, or constraint that biases the angle toward a target angular value or range of values during simulation. The angle restraint can be implemented using one or more functions and can be static or dynamically modified during one or more stages of a molecular dynamics simulation, including during particular lambda windows.

As used herein, the term “dihedral restraint” refers to a positional restraint configured to regulate or influence a dihedral angle defined by four atoms within a molecular dynamics model. In some embodiments, a dihedral restraint applies a potential, force, or constraint that biases the dihedral angle toward a target torsional value or range of values during simulation. The dihedral restraint can be implemented using one or more functions and can be static or dynamically modified during one or more stages of a molecular dynamics simulation, including during particular lambda windows.

In one or more embodiments, the free energy optimization system 100 generates a plurality of positional restraints for a restraint atom combination by generating multiple angle restraints and multiple dihedral restraints corresponding to different geometric relationships between the ligand and the protein. For instance, the free energy optimization system 100 can generate a first angle restraint and a second angle restraint associated with the selected restraint atoms and can generate a first dihedral restraint, a second dihedral restraint, and a third dihedral restraint associated with the selected restraint atoms. In some embodiments, the free energy optimization system 100 stores the generated positional restraints as restraint parameters for execution within the molecular dynamics model. The free energy optimization system 100 can then provide the restraint parameters to the molecular dynamics model to enable application of the positional restraints during simulation.

In addition to angle restraints and dihedral restraints, the free energy optimization system 100 can also generate distance restraints. As used herein, the term “distance restraint” refers to a positional restraint configured to regulate or influence a distance between two atoms within a molecular dynamics model. In some embodiments, a distance restraint applies a potential, force, or constraint that biases the distance between a ligand atom and a protein atom toward a target value or range of values during simulation. The distance restraint can be implemented using one or more functions and can be static or dynamically modified during one or more stages of a molecular dynamics simulation, including during particular lambda windows.

In one or more embodiments, the free energy optimization system 100 generates a distance restraint by defining a target distance between two selected restraint atoms and associating the target distance with one or more restraint parameters for execution within the molecular dynamics model. In particular, the free energy optimization system 100 can determine the target distance based on a reference configuration of the ligand-protein complex or based on distance values observed during simulation. The distance restraint can thereby regulate separation between the selected ligand atom and protein atom during simulation. In some embodiments, the distance restraint is generated in combination with one or more angle restraints and one or more dihedral restraints to define a multi-parameter positional restraint configuration between the ligand and the protein.

As previously mentioned, in one or more embodiments the free energy optimization system 100 applies positional restraints during execution of a molecular dynamics model to generate a binding free energy between a ligand and a protein. In particular, the free energy optimization system 100 applies the positional restraints during lambda windows of the molecular dynamics model while annihilating one or more nonbonded interactions between the ligand and the protein in accordance with lambda parameters. FIGS. 5A-5B illustrate schematic diagrams of a free energy optimization system 100 applying restraints during lambda windows of a molecular dynamics model in accordance with one or more embodiments. Specifically, FIG. 5A illustrates an overview of the free energy optimization system 100 sequentially annihilating nonbonded interactions and applying positional restraints, including dihedral restraints, angle restraints, and an angular stabilization potential. FIG. 5B illustrates a graph depicting an example lambda schedule showing progression of lambda parameters associated with annihilating nonbonded interactions and applying dihedral and angle restraints during execution of a molecular dynamics model.

As illustrated in FIG. 5A, in some embodiments the free energy optimization system 100 performs a step 502 of annihilating a nonbonded interaction. As used herein, the term “nonbonded interaction” refers to an interaction between atoms that are not directly connected by a bonded interaction within a molecular topology. For instance, nonbonded interaction refers to an interaction between atoms that are not connected to each other directly with a covalent bond. In some embodiments, nonbonded interactions can include electrostatic interactions, Lennard-Jones interactions, van der Waals interactions, dispersion forces, steric repulsion, or combinations thereof. Nonbonded interactions can be modeled using one or more potential energy functions within a molecular dynamics simulation and can be selectively scaled, modified, decoupled, or annihilated during an alchemical transformation.

In one or more embodiments, the free energy optimization system 100 annihilates a first type of nonbonded interaction between the ligand and the protein. In particular, the free energy optimization system 100 progressively reduces interaction parameters associated with the first type of nonbonded interaction across a plurality of lambda windows of the molecular dynamics model. As used herein, the term “lambda windows” refers to staged values of an alchemical parameter that control modification of interaction terms within a molecular dynamics simulation during a free energy calculation.

In some embodiments, the first type of nonbonded interaction corresponds to electrostatic interactions between the ligand and the protein, such that electrostatic (e.g., Coulomb) interaction parameters are reduced toward zero during an initial subset of lambda windows of an alchemical transformation. In some cases, the first type of nonbonded interaction is substantially annihilated prior to annihilation of a second type of nonbonded interaction. In addition, in one or more embodiments, the free energy optimization system 100 generates a set of lambda windows for annihilating the second type of nonbonded interactions by generating free energy estimation scores and selecting lambda windows based on variance between the free energy estimation scores, as described further below in relation to FIG. 6.

As further illustrated, in one or more embodiments the free energy optimization system 100 performs a step 504 of applying dihedral restraints. In particular, the free energy optimization system 100 applies the dihedral restraints during a first set of lambda windows of the molecular dynamics model (e.g., the first 32 lambda windows). In some cases, the dihedral restraints are introduced concurrently with annihilation of the first type of nonbonded interaction. For example, the free energy optimization system 100 can introduce one or more dihedral restraints during an initial subset of lambda windows while electrostatic interaction parameters are being reduced.

For example, dihedral restraints include constraints applied to a torsion (dihedral) angle defined by four atoms (A-B-C-D) to keep that angle near a specified value during a molecular simulation. Thus, as illustrated in step 504, a dihedral angle can be defined by a first atom of the protein, a first atom of the ligand, a second (adjacent or connected) atom of the ligand, and a third (adjacent or connected) atom of the ligand. Similarly, a dihedral angle can be defined by a first atom of the protein, a second atom of the protein, a first atom of the ligand, and a second atom of the ligand. Moreover, a dihedral angle can be defined by a first atom of the protein, a second atom of the protein, a third atom of the protein, and a first atom of the ligand.

As further shown in FIG. 5A, the free energy optimization system 100 performs a step 506 of annihilating a second type of nonbonded interaction between the ligand and the protein. In some embodiments, the second type of nonbonded interaction corresponds to Lennard-Jones (LJ) interactions between the ligand and the protein. For example, the free energy optimization system 100 can progressively reduce parameters associated with the second type of nonbonded interaction across a subsequent plurality of lambda windows. In some cases, the free energy optimization system 100 annihilates the second type of nonbonded interactions after annihilating the first type of nonbonded interaction.

In one or more embodiments, the free energy optimization system 100 varies ordering of annihilation of different types of nonbonded interactions. For example, electrostatic interactions can be reduced prior to reduction of Lennard-Jones interactions, Lennard-Jones interactions can be reduced prior to reduction of electrostatic interactions, or reductions can occur partially concurrently across overlapping lambda windows. The free energy optimization system 100 can select an ordering or degree of overlap based on statistical free energy estimation metrics, configurational overlap behavior, or predefined protocol parameters.

In one or more embodiments, during or following annihilation of the first and second types of nonbonded interactions, the free energy optimization system 100 configures an alchemical end state corresponding to a modified or dummy ligand state within the molecular dynamics model. For example, as parameters for the first type of nonbonded interaction and the second type of nonbonded interaction (e.g., electrostatic interaction parameters and Lennard-Jones epsilon parameters) associated with the ligand are progressively reduced across lambda windows, the ligand transitions toward a state in which nonbonded interactions with the protein are substantially removed. In certain embodiments, intramolecular nonbonded interactions of the ligand (e.g., 1-4 electrostatic or Lennard-Jones interactions) can be selectively retained or modified depending on configuration parameters of the molecular dynamics framework. Such configuration defines the alchemical dummy state used in generating the binding free energy.

In one or more embodiments, the free energy optimization system 100 also modifies one or more internal dihedrals of the ligand during annihilation by scaling associated torsional force constants toward zero as a function of lambda during progression toward the alchemical dummy state. In particular, the free energy optimization system 100 flattens internal dihedrals (e.g., rotatable torsions) within the ligand by scaling associated torsional force constants toward zero as a function of lambda, while multiplicity and phase parameters remain unchanged. For example, torsions corresponding to predefined chemical patterns are identified and modified, while nonrotatable dihedrals, bonds, and angles remain unaltered. Such modifications can facilitate a well-defined alchemical dummy state while preserving stability of the molecular topology.

In one or more embodiments, the free energy optimization system 100 identifies rotatable torsions of the ligand using predefined chemical pattern recognition rules. For example, the free energy optimization system 100 can evaluate chemical substructures of the ligand using pattern-matching expressions (e.g., SMARTS patterns or equivalent structural descriptors) to identify single bonds between non-terminal heavy atoms that are not part of ring systems, amide linkages, or other chemically constrained motifs. The free energy optimization system 100 can selectively scale torsional force constants associated with identified rotatable torsions toward zero as a function of lambda while retaining multiplicity and phase parameters, thereby flattening internal torsional energy barriers while preserving bonded topology of the ligand.

In addition, in one or more embodiments the free energy optimization system 100 performs a step 508 of applying angle restraints. In particular, the free energy optimization system 100 applies the angle restraints during a second set of lambda windows of the molecular dynamics model (e.g., during the final 32 lambda windows). In some embodiments, the angle restraints are introduced during a later subset of lambda windows that overlap with annihilation of the second type of nonbonded interaction.

In addition, in some embodiments the free energy optimization system 100 also applies one or more distance restraints while applying the angle restraints. In particular, the free energy optimization system 100 applies one or more distance restraints in conjunction with the angle restraints to regulate translational separation between the ligand and the protein as nonbonded interactions are reduced. Together, the distance restraint and angle restraints define positional relationships that maintain orientation and positioning of the ligand relative to the protein during progression toward the alchemical end state.

As further illustrated, in some embodiments the free energy optimization system 100 performs a step 510 of applying an angular stabilization potential to one or more angle restraints during the lambda windows of the molecular dynamics model. As used herein, the term “angular stabilization potential” refers to a potential applied to an angle restraint that is configured to regulate angular behavior near geometrically unstable configurations. In some embodiments, the angular stabilization potential increases a restorative force as an angle approaches a threshold angular geometry, including geometries that approach collinearity. The angular stabilization potential can be implemented using one or more nonlinear, bounded, or otherwise modified potential energy functions that prevent numerical instability or excessive force behavior within a molecular dynamics simulation. In some cases, the angular stabilization potential is a restricted bending potential, including a restricted bending potential of the type implemented in molecular dynamics engines (e.g., GROMACS).

In some embodiments, the angular stabilization potential comprises a piecewise or nonlinearly bounded potential energy function that includes a relatively flat region within a predefined operational angular range and a progressively increasing gradient as the angle approaches a threshold angular geometry. For example, the angular stabilization potential can be configured such that, for angular values within a mid-range interval, a harmonic-like potential is applied, and for angular values approaching near-collinear configurations (e.g., approaching 0° or 180°), the potential energy increases more rapidly than a purely harmonic potential, thereby limiting approach to singular geometric configurations while maintaining bounded derivatives suitable for numerical integration within the molecular dynamics model.

In some embodiments, the free energy optimization system 100 applies an angular stabilization potential that increases a restorative force as an angle approaches a threshold angular geometry, thereby reducing likelihood of near-collinear configurations during partial or full annihilation of nonbonded interactions. In particular, the angular stabilization potential can be applied to one or more angle restraints generated from the selected restraint atoms and can modify a restraint potential as the monitored angle approaches a predefined threshold value corresponding to a near-collinear configuration (e.g., approaching 0° or 180°). In some cases, the angular stabilization potential is configured such that the magnitude of the restorative force increases nonlinearly as the angle approaches the threshold angular geometry, thereby discouraging configurations that may lead to numerical instability or ill-conditioned geometric relationships within the molecular dynamics model. In addition, in some instances, the angular stabilization potential is selectively activated during one or more lambda windows in which nonbonded interactions are being reduced, thereby maintaining geometric stability of the ligand-protein complex during progression toward an alchemical end state.

FIG. 5B illustrates an example graph 512 depicting an example progression of lambda parameters associated with annihilating nonbonded interactions and applying positional restraints across lambda windows of a molecular dynamics model. The illustrated schedule in FIG. 5B represents one example arrangement, and alternative lambda window configurations can be utilized in accordance with one or more embodiments. For example, as shown, the free energy optimization system 100 can also apply dihedral restraints 518 during a first set of lambda windows (e.g., the first 32 lambda windows) and concurrently applying distance restraints and angle restraints 522 and also concurrently flattening internal dihedrals 520. As also shown, the free energy optimization system 100 can annihilate a first type of nonbonded interaction 514 while concurrently applying dihedral restraints 518. In addition, the free energy optimization system 100 reduces lambda parameters corresponding to a first type of nonbonded interaction 514 (e.g., electrostatic interactions) prior to reduction of lambda parameters corresponding to a second type of nonbonded interaction 516 (e.g., Lennard-Jones interactions).

As previously mentioned, in one or more embodiments the free energy optimization system 100 generates lambda windows for annihilating a type of nonbonded interaction during execution of a molecular dynamics model. In particular, the free energy optimization system 100 evaluates candidate sets of lambda windows using a statistical free energy estimation algorithm and selects a set of lambda windows based on variance between corresponding free energy estimation scores. FIG. 6 illustrates a schematic diagram of a free energy optimization system selecting an optimized set of lambda windows for annihilating a type of nonbonded interaction in accordance with one or more embodiments.

As used herein, the term “lambda windows” refers to staged values of an alchemical coupling parameter used to control scaling or modification of one or more interaction terms within a molecular dynamics simulation. In some embodiments, lambda windows correspond to discrete or continuous parameter values that modulate nonbonded interactions, bonded interactions, restraints, or combinations thereof during a free energy calculation. Lambda windows can define intermediate states of an alchemical transformation between physical and alchemical end states.

As illustrated in FIG. 6, the free energy optimization system 100 performs a step 602 of selecting candidate lambda windows. In particular, the free energy optimization system 100 generates or identifies a candidate set of lambda windows defining staged parameter values for annihilating a type of nonbonded interaction within a molecular dynamics model. In some embodiments, the candidate lambda windows can be uniformly spaced, non-uniformly spaced, or arranged according to an initial heuristic, predefined schedule, or prior simulation data.

In one or more embodiments, the free energy optimization system 100 generates a reference dense schedule of lambda windows comprising a first plurality of lambda windows having a higher resolution than a target execution schedule. For example, the reference dense lambda schedule can include at least 100 lambda windows, at least 150 lambda windows, at least 192 lambda windows, or another dense sampling of lambda values spanning a coupling parameter from an initial value to a final value. The free energy optimization system 100 can evaluate statistical behavior across the reference dense lambda schedule and subsequently generate a reduced execution schedule by selecting a subset of lambda windows from the reference dense lambda schedule based on one or more statistical free energy estimation criteria. In some embodiments, the reduced execution schedule comprises fewer than half of the reference dense lambda windows, such as selecting approximately 44 lambda windows from approximately 192 lambda windows, although other ratios can be utilized.

As further illustrated, the free energy optimization system 100 performs a step 604 of generating free energy estimation scores for candidate lambda windows in an interactive loop. In particular, the free energy optimization system 100 iteratively evaluates candidate sets of lambda windows using a statistical free energy estimation algorithm to assess performance of the candidate sets during annihilation of the type of nonbonded interaction.

As used herein, the term “statistical free energy estimation algorithm” refers to a computational method configured to estimate a free energy difference between states of a molecular system based on statistical analysis of simulation data generated from a molecular dynamics model. In some embodiments, the statistical free energy estimation algorithm utilizes sampled potential energy values, reduced potentials, overlap metrics, probability distributions, or combinations thereof associated with multiple lambda windows to compute a free energy estimate. Examples of statistical free energy estimation algorithms include multistate Bennett acceptance ratio (MBAR), Bennett acceptance ratio (BAR), weighted histogram analysis method (WHAM), thermodynamic integration techniques, or related estimators.

In one or more embodiments, the statistical free energy estimation algorithm generates overlap metrics that quantify configurational overlap between adjacent or non-adjacent lambda windows. For example, the free energy optimization system 100 can compute reduced potential overlap matrices, pairwise state overlap values, cross-state reweighting efficiency metrics, or related statistical coupling measures between lambda windows. The free energy optimization system 100 can utilize these overlap metrics to identify regions of insufficient configurational overlap and relocate or insert lambda windows in those regions to improve estimator stability and reduce statistical uncertainty.

As used herein, the term “free energy estimation score” refers to a quantitative output generated by a statistical free energy estimation algorithm based on simulation data corresponding to one or more lambda windows. In some embodiments, a free energy estimation score can correspond to an estimated free energy difference, an uncertainty value associated with the estimated free energy difference, a variance metric (e.g., gradient), or another statistical measure derived from sampled configurations across lambda windows. A free energy estimation score can be used to evaluate performance, stability, convergence, or reliability of a candidate set of lambda windows during an alchemical transformation.

As mentioned, the free energy optimization system 100 can generate free energy estimation scores for candidate lambda windows in an interactive loop. In particular, the free energy optimization system 100 generates the free energy estimation scores by performing a step 606 of generating a first free energy estimation score for a candidate set of lambda windows, then modifying the candidate lambda windows and performing a step 608 of generating a second free energy estimation score for the modified candidate set of lambda windows. In some embodiments, the free energy optimization system 100 generates the first and second free energy estimation scores by executing molecular dynamics simulations across the respective lambda windows and applying a statistical free energy estimation algorithm to simulation data generated at the lambda windows. The modification of the candidate lambda windows can include adjusting spacing between lambda values, redistributing lambda windows across regions associated with higher free energy curvature, increasing or decreasing a number of lambda windows, or otherwise altering staging of the alchemical coupling parameter. The free energy optimization system 100 can iteratively repeat generation and modification of candidate lambda windows within the interactive loop to produce multiple free energy estimation scores corresponding to different lambda window configurations.

In one or more embodiments, the free energy optimization system 100 modifies the candidate lambda windows by iteratively relocating one or more lambda window positions along the coupling parameter axis based on a gradient or sensitivity metric derived from the statistical free energy estimation algorithm. For example, the free energy optimization system 100 can compute a local sensitivity of an estimated free energy difference with respect to a change in a lambda window position and shift one or more lambda windows toward regions exhibiting increased statistical variance, reduced configurational overlap, or elevated curvature of the free energy surface. In certain implementations, the free energy optimization system 100 performs multiple iterations of relocating lambda windows, regenerating free energy estimation scores after each relocation, and evaluating convergence criteria prior to selecting a final set of lambda windows for execution.

In some embodiments, the free energy optimization system 100 determines that convergence has been achieved based on one or more predefined convergence criteria. For example, convergence criteria can include a condition that changes in free energy estimation scores between successive iterations fall below a threshold value, that a variance metric across adjacent lambda windows satisfies a predefined tolerance, that gradient values associated with lambda relocation fall below a threshold magnitude, or that configurational overlap metrics between adjacent lambda windows exceed a minimum overlap threshold. Upon satisfaction of the convergence criteria, the free energy optimization system 100 selects the then-current lambda window configuration as the optimized set of lambda windows for annihilating the type of nonbonded interaction.

As further illustrated in FIG. 6, the free energy optimization system 100 performs a step 610 of optimizing variance of free energy estimation scores. In particular, the free energy optimization system 100 determines a quantitative variance between the first free energy estimation score generated for the candidate set of lambda windows and the second free energy estimation score generated for the modified candidate set of lambda windows. In some embodiments, the variance reflects a statistical difference, dispersion, or uncertainty between estimated free energy values derived from molecular dynamics simulation data corresponding to the respective lambda window configurations. For example, the variance can correspond to a difference in estimated free energy values, a difference in associated uncertainty metrics, or another statistical measure generated by the statistical free energy estimation algorithm. The free energy optimization system 100 evaluates the determined variance to assess relative statistical behavior of the candidate and modified candidate lambda window configurations during annihilation of the type of nonbonded interaction (e.g., the second type of nonbonded interaction).

As further illustrated in FIG. 6, the free energy optimization system 100 performs a step 612 of selecting a set of lambda windows for annihilating the type of nonbonded interaction based on the variance between the first free energy estimation score and the second free energy estimation score. In particular, the free energy optimization system 100 selects one of the candidate set of lambda windows or the modified candidate set of lambda windows based on the determined variance. In some embodiments, the free energy optimization system 100 selects the set of lambda windows associated with a reduced variance, lower uncertainty, or otherwise improved statistical behavior relative to other evaluated lambda window configurations. The selected set of lambda windows is then utilized during execution of the molecular dynamics model to perform annihilation of the type of nonbonded interaction as part of generating a binding free energy between the ligand and the protein. In some cases, the free energy optimization system 100 utilizes the set of lambda windows for annihilating the second type of nonbonded interaction.

As previously mentioned, the free energy optimization system 100 improves accuracy of over conventional systems. In particular, the free energy optimization system 100 reduces variability across replicate molecular dynamics cycles when generating binding free energies, thereby producing more consistent and reproducible free energy estimates. FIG. 7 illustrates a graph depicting the free energy optimization system generating binding free energies for known molecular compounds for known ligands with improved variability across replicate molecular dynamics cycles in accordance with one or more embodiments.

FIG. 7 illustrates a graph 700 depicting the free energy optimization system 100 generating binding free energies for known molecular compounds across multiple protein targets with improved consistency across replicate molecular dynamics cycles, as reflected by reduced standard deviation values. As shown, the graph 700 compares variability in binding free energy calculations across different targets, demonstrating lower dispersion in standard deviation values for the free energy optimization system 100 when applying the disclosed protocol relative to conventional systems. Indeed, the reduced variability indicates enhanced reproducibility and improved convergence behavior of the free energy optimization system 100.

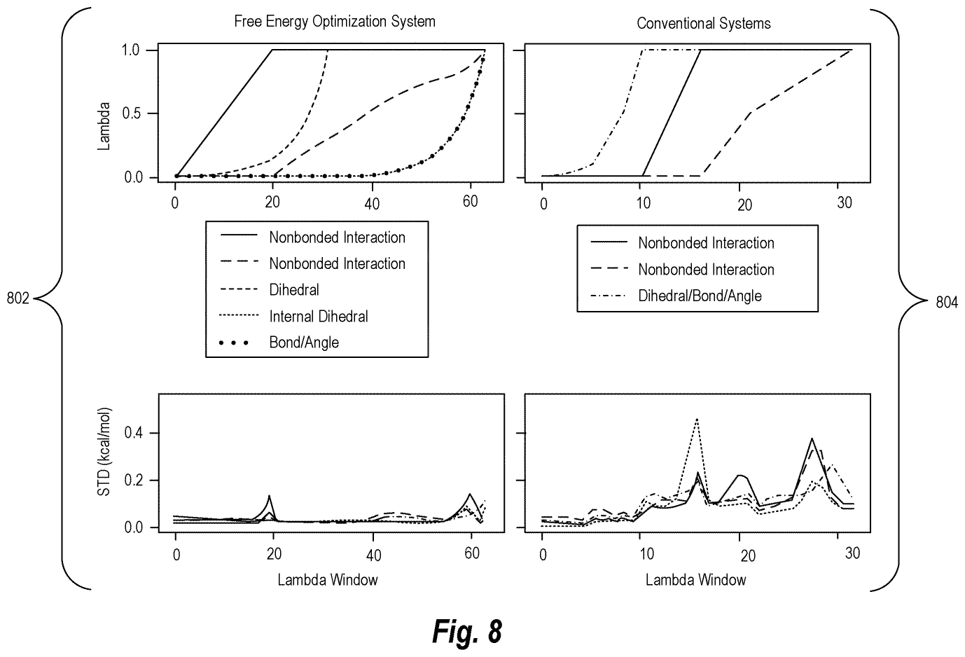

As previously mentioned, the free energy optimization system 100 improves stability of binding free energy calculations across lambda windows. In particular, the free energy optimization system 100 reduces variability that can arise during staged annihilation of nonbonded interactions by applying geometry-conditioned positional restraints and optimized lambda window configurations. FIG. 8 illustrates graphs depicting reduced variability across lambda windows for binding free energy calculations, demonstrating improved stability of thermodynamic transformations relative to conventional systems, in accordance with one or more embodiments.

FIG. 8 illustrates graphs 802 depicting the free energy optimization system 100 reducing variability across lambda windows during staged annihilation of nonbonded interactions in generating a binding free energy in comparison to graphs 804 for conventional systems. As shown, the graphs 802 and the graphs 804 compare standard deviation values across individual lambda windows during alchemical transformation stages, including lambda windows corresponding to progressive scaling of nonbonded interactions. As shown in graphs 804, in conventional interaction-scaling configurations, variability increases at certain intermediate lambda windows, reflecting instability during portions of the thermodynamic transformation. By contrast, as shown in graphs 802, the free energy optimization system 100 maintains lower and more uniform standard deviation values across the lambda windows when applying geometry-conditioned positional restraints and optimized lambda window configurations. Indeed, the reduced variability across intermediate transformation stages indicates improved stability of the thermodynamic cycle and enhanced consistency of binding free energy estimation for the free energy optimization system 100.

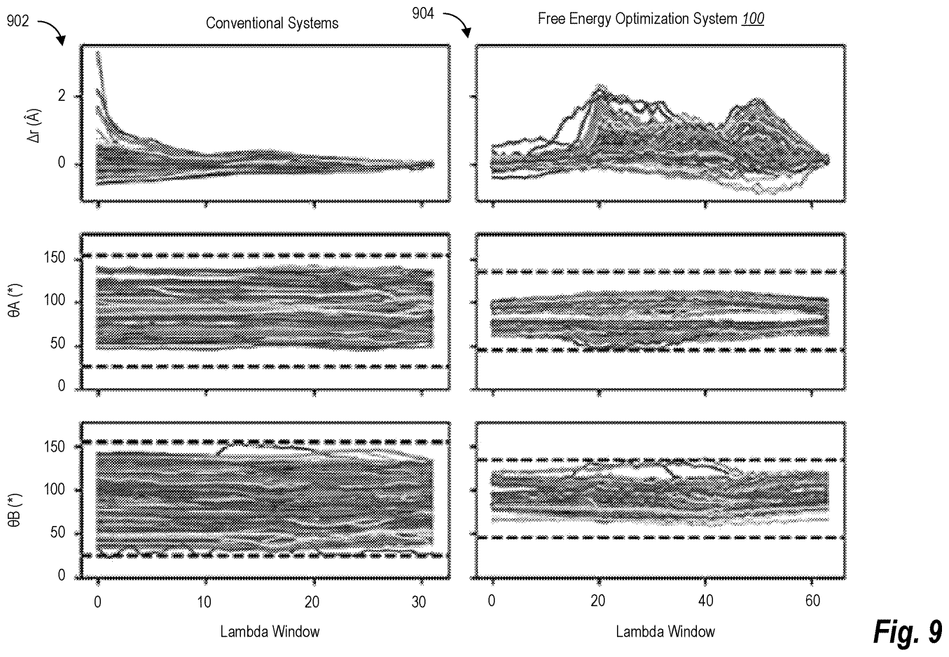

As previously mentioned, the free energy optimization system 100 improves the reliability and robustness of binding free energy calculations relative to conventional systems. In particular, the free energy optimization system 100 reduces fluctuation and instability across thermodynamic transformation stages by applying geometry-conditioned positional restraints and optimized lambda window configurations. FIG. 9 illustrates graphs depicting the free energy optimization system reducing variability between adjacent lambda windows during an alchemical transformation in accordance with one or more embodiments.

FIG. 9 illustrates graphs depicting interaction energy behavior across lambda windows for different interaction-scaling configurations during binding free energy calculations. As shown, the graphs 902 depict energy profiles associated with staged annihilation of nonbonded interactions across multiple simulations for conventional systems, while graphs 904 depict energy profiles associated with staged annihilation of nonbonded interactions across multiple simulations for conventional systems for the free energy optimization system 100. As shown in graphs 902, conventional systems exhibit increased fluctuation and broader dispersion of energy values across certain lambda windows, particularly during intermediate stages of the transformation. By contrast, as shown in graphs 902, the free energy optimization system 100 maintains more controlled and uniform energy profiles across the lambda windows when applying the disclosed positional restraint and lambda window configuration protocol. Indeed, the reduced fluctuation and tighter energy distributions reflect enhanced thermodynamic stability and improved consistency of binding free energy estimation for the free energy optimization system 100.

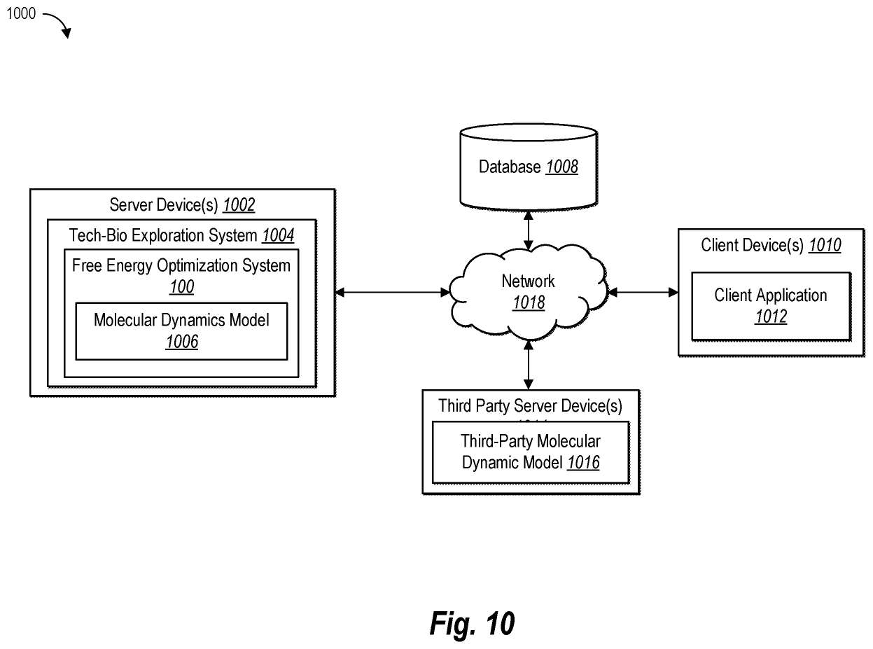

As previously mentioned, the free energy optimization system generates a binding free energy that selects positional restraints and configures lambda windows within a molecular dynamics model to generate binding free energies for ligand-protein systems. In particular, the free energy optimization system 100 utilizes various devices, servers, and networks for storing, synchronizing, and communicating regarding simulation inputs, intermediate computational data, restraint parameters, lambda window configurations, and binding free energy results. FIG. 10 illustrates a schematic diagram of an environment in which a free energy optimization system 100 can operate in accordance with one or more embodiments.

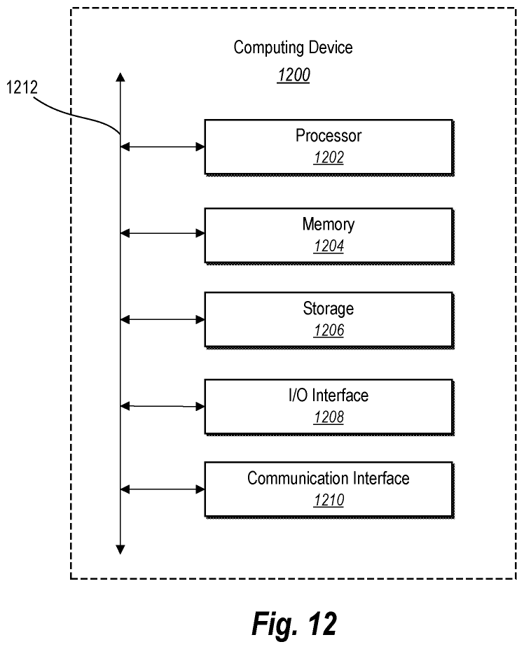

As shown, the environment 1000 includes server device(s) 1002, database 1008, client device(s) 1010, and third-party server device(s) 1014. Each of the components of the environment 1000 can communicate via network 1018 and network 1018 may be any suitable network over which computing devices can communicate. Example networks are discussed in more detail in relation to FIG. 12.

As mentioned above, the environment 1000 includes client device(s) 1010. The client device(s) 1010 can be one of a variety of computing devices, including a smartphone a tablet, a smart television, a desktop computer, a laptop computer, a virtual reality device, an augmented reality device, or another computing device as described in relation to FIG. 12. The client device(s) 1010 can communicate with the server device(s) 1002 via network 1018. For example, the client device(s) 1010 can receive user input from a user interacting with client device(s) 1010 (e.g., via the client application(s) 1012) to, for instance, receive user interactions regarding candidate ligands and/or candidate proteins. In addition, the free energy optimization system or the server device(s) 1002 can receive information relating to various interactions with various user interface elements based on the input received by the client device(s) 1010.

As shown, the client device(s) 1010 can include a client application(s) 1012. In particular, the client application(s) 1012 may be a web application, a native application installed on the client device(s) 1010 (e.g., a mobile application, a desktop application, etc.), or a cloud-based application where all or part of the functionality is performed by the server device(s) 1002. Based on instructions from the client application(s) 1012, the client device(s) 1010 can present or display information, including a user interface for receiving user input regarding candidate ligands and/or candidate proteins. Using the client application(s) 1012, the client device(s) 1010 can perform (or request to perform) various operations, such as displaying information regarding binding free energies, selected restraint atoms, and/or various components or phases of a thermodynamic cycle for generating binding free energies.

As illustrated in FIG. 10, the environment 1000 also includes the server device(s) 1002. The server device(s) 1002 may generate, track, store, process, receive, and transmit electronic data, such as binding free energies, data corresponding to chemical or molecular structures for candidate ligands and/or candidate proteins, candidate atom information, positional restraint parameters, lambda window configurations, loss function metrics, molecular dynamics simulation data, and interactions between user accounts or client devices. For example, the server device(s) 1002 may receive an indication from the client device(s) 1010 of a user interaction selecting (or inputting) candidate ligands and/or candidate proteins. In addition, the server device(s) 1002 can transmit data to the client device(s) 1010 in the form of a binding free energy. Indeed, the server device(s) 1002 can communicate with the client device(s) 1010 to send and/or receive data via network 1018. In some implementations, the server device(s) 1002 comprise(s) a distributed server where the server device(s) 1002 include(s) a number of server devices distributed across the network 1018 and located in different physical locations. The server device(s) 1002 can comprise one or more content servers, application servers, container orchestration servers, communication servers, web-hosting servers, machine learning servers, and other types of servers.

As shown in FIG. 10, the server device(s) 1002 can also include the free energy optimization system 100 as part of the tech-bio exploration system 1004. The tech-bio exploration system 1004 can communicate with the client device(s) 1010 to perform various functions associated with the client application(s) 1012, such as receiving user input identifying candidate ligands and/or candidate proteins, retrieving and managing molecular structure data, coordinating molecular dynamics simulations, invoking the free energy optimization system to generate positional restraints and configure lambda windows, aggregating binding free energy results, and presenting analytical outputs to users via the client application(s) 1012. Indeed, the tech-bio exploration system 1004 can include a network-based smart cloud storage system to manage, store, and maintain binding free energy data, molecular dynamics simulation data, positional restraint parameters, lambda window configurations, molecular structure data for candidate ligands and proteins, loss function evaluation data, and related computational results across numerous user accounts. In some embodiments, the free energy optimization system and/or the tech-bio exploration system 1004 utilize the database 1008 to store and access information such as molecular structure files, candidate atom selections, restraint definitions, lambda window schedules, simulation outputs, and/or binding free energy estimates.

As further shown in FIG. 10, the free energy optimization system 100 (or the tech-bio exploration system 1004) can host a molecular dynamics model 1006. The molecular dynamics model 1006 can interface with the free energy optimization system 100 to receive positional restraint parameters, lambda window configurations, and other simulation inputs generated based on candidate ligands and candidate proteins. The molecular dynamics model 1006 may execute thermodynamic transformations across the configured lambda windows to simulate interactions between the ligand and the protein and generate simulation trajectory data, energy data, and intermediate computational results. The generated simulation data may be stored in the database 1008, processed by the free energy optimization system 100 to compute binding free energy estimates, and transmitted via the server device(s) 1002 to the client device(s) 1010 for presentation through the client application(s) 1012.

As further illustrated in FIG. 10, the environment 1000 may also include third-party server device(s) 1014 hosting a third-party molecular dynamics model 1016. In some embodiments, the free energy optimization system 100 (and/or the tech-bio exploration system 1004) can interface with the third-party molecular dynamics model 1016 to transmit positional restraint parameters, lambda window configurations, and other simulation inputs for execution of molecular dynamics simulations. The third-party molecular dynamics model 1016 may execute thermodynamic transformations across the configured lambda windows, as requested by the free energy optimization system 100, and return simulation trajectory data, energy values, and related computational outputs to the server device(s) 1002 for further processing. The free energy optimization system 100 may then utilize the returned simulation data to compute binding free energy estimates, store simulation outputs in the database 1008, and transmit results to the client device(s) 1010 via the network 1018.

Although FIG. 10 depicts the free energy optimization system located on the server device(s) 1002, in some implementations, the free energy optimization system may be implemented by (e.g., located entirely or in part on) one or more other components of the environment. For example, the free energy optimization system may be implemented as part of client device(s) 1010 and/or a third-party system. As another example, the client device(s) 1010 and/or a third-party system can download all or part of the free energy optimization system for implementation independent of, or together with, the server device(s) 1002.

In some implementations, though not illustrated in FIG. 10, the environment 1000 may have a different arrangement of components and/or may have a different number or set of components altogether. For example, the client device(s) 1010 may communicate directly with the free energy optimization system, bypassing network 1018. The environment 1000 may also include one or more third-party systems, each corresponding to a different data source. In addition, the environment 1000 can include the database 1008 located external to the server device(s) 1002 (e.g., in communication via the network 1018) or located on the server device(s) 1002 and/or on the client device(s) 1010.

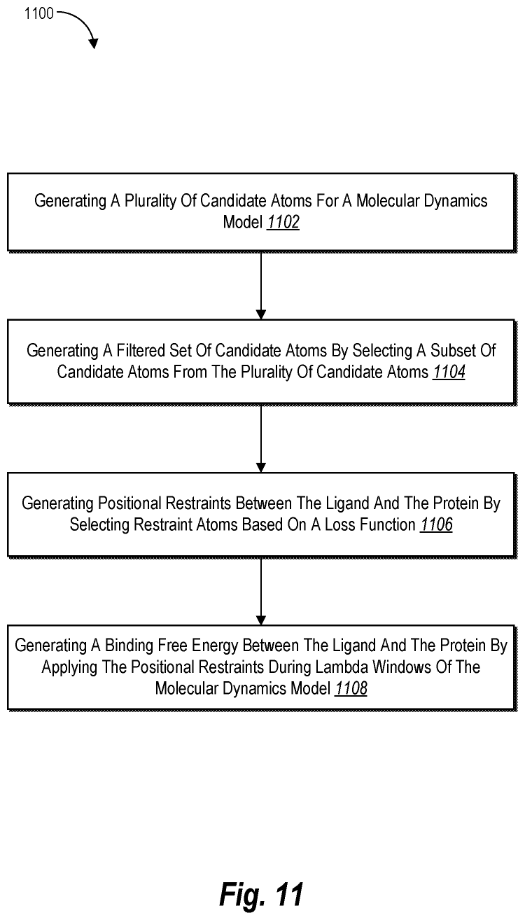

FIGS. 1-10, the corresponding text, and the examples provide a number of different methods, systems, devices, and non-transitory computer-readable media of the free energy optimization system. In addition to the foregoing, one or more embodiments can also be described in terms of flowcharts comprising acts for accomplishing a particular result, as shown in FIG. 11. FIG. 11 may be performed with more or fewer acts. Further, the acts may be performed in differing orders. Additionally, the acts described herein may be repeated or performed in parallel with one another or parallel with different instances of the same or similar acts.

As mentioned, FIG. 11 illustrates a flowchart of a series of acts 1100 for improving binding free energy calculations in molecular dynamics simulations in accordance with one or more embodiments. While FIG. 11 illustrates acts according to one embodiment, alternative embodiments may omit, add to, reorder, and/or modify any of the acts shown in FIG. 11. The acts of FIG. 11 can be performed as part of a method. Alternatively, a non-transitory computer-readable medium can comprise instructions that, when executed by one or more processors, cause a computing device to perform the acts of FIG. 11. In some embodiments, a system can perform the acts of FIG. 11.

As shown in FIG. 11, the series of acts 1100 includes an act 1102 of generate a plurality of candidate atoms for a molecular dynamics model, an act 1104 of generating a filtered set of candidate atoms by selecting a subset of candidate atoms from the plurality of candidate atoms, an act 1106 of generating positional restraints between the ligand and the protein by selecting restraint atoms based on a loss function, and an act 1108 of generating a binding free energy between the ligand and the protein by applying the positional restraints during lambda windows of the molecular dynamics model.

In particular, the act 1102 can include generating, based on simulated binding interactions between a ligand and a protein, a plurality of candidate atoms for a molecular dynamics model, the act 1104 can include generating a filtered set of candidate atoms by selecting a subset of candidate atoms from the plurality of candidate atoms based on angle metrics between the ligand and the protein for the subset of candidate atoms satisfying an angle metric threshold, the act 1106 can include generating positional restraints between the ligand and the protein by selecting, from the filtered set of candidate atoms, restraint atoms based on a loss function that combines a measure of angular variability that penalizes angular variability with an angular conditioning factor that penalizes collinear geometry, and the act 1108 can include generating a binding free energy between the ligand and the protein by applying the positional restraints during lambda windows of the molecular dynamics model.

For example, the series of acts 1100 can include acts to perform any of the operations described in the following clauses:

CLAUSE 1. A computer-implemented method comprising generating, based on simulated binding interactions between a ligand and a protein, a plurality of candidate atoms for a molecular dynamics model, generating a filtered set of candidate atoms by selecting a subset of candidate atoms from the plurality of candidate atoms based on angle metrics between the ligand and the protein for the subset of candidate atoms satisfying an angle metric threshold, generating positional restraints between the ligand and the protein by selecting, from the filtered set of candidate atoms, restraint atoms based on a loss function that combines a measure of angular variability that penalizes angular variability with an angular conditioning factor that penalizes collinear geometry, and generating a binding free energy between the ligand and the protein by applying the positional restraints during lambda windows of the molecular dynamics model.

CLAUSE 2. The computer-implemented method of clause 1, further comprising generating the positional restraints between the ligand and the protein by selecting the restraint atoms based on the loss function that combines the measure of angular variability and the angular conditioning factor with a measure of distance that penalizes distance between candidate atoms and a measure of distance variability that penalizes distance variability.

CLAUSE 3. The computer-implemented method of any of clauses 1-2, further comprising generating the positional restraints between the ligand and the protein by selecting the restraint atoms based on the loss function that combines a first measure of angular variability for a first angle for the restraint atoms and a second measure of angle variability for a second angle for the restraint atoms.

CLAUSE 4. The computer-implemented method of any of clauses 1-3, further comprising generating the positional restraints between the ligand and the protein by selecting the restraint atoms based on the loss function that combines a first measure of dihedral angular variability for the restraint atoms, a second measure of dihedral angular variability for the restraint atoms, and a third measure of dihedral angular variability for the restraint atoms.

CLAUSE 5. The computer-implemented method of any of clauses 1-4, further comprising applying an angular stabilization potential to an angle restraint of the positional restraints during the lambda windows of the molecular dynamics model, wherein the angular stabilization potential is configured to increase a restorative force as the angle restraint approaches a threshold angular geometry.

CLAUSE 6. The computer-implemented method of any of clauses 1-5, further comprising applying the positional restraints to the lambda windows of the molecular dynamics model by: applying the positional restraints during the lambda windows by applying dihedral restraints during a first set of lambda windows of the molecular dynamics model, and applying the positional restraints during the lambda windows by applying angle restraints during a second set of lambda windows of the molecular dynamics model.

CLAUSE 7. The computer-implemented method of any of clauses 1-6, further comprising annihilating a first type of nonbonded interaction concurrently with applying dihedral restraints during the lambda windows of the molecular dynamics model.

CLAUSE 8. The computer-implemented method of any of clauses 1-7, further comprising optimizing a set of lambda windows for annihilating a type of nonbonded interaction by: determining, utilizing a statistical free energy estimation algorithm, a first free energy estimation score for a candidate set of lambda windows, determining, utilizing the statistical free energy estimation algorithm, a second free energy estimation score for a modified candidate set of lambda windows, and selecting the set of lambda windows for annihilating the type of nonbonded interaction based on a variance between the first free energy estimation score and the second free energy estimation score.

CLAUSE 9. The computer-implemented method of any of clauses 1-8, wherein generating the plurality of candidate atoms for the molecular dynamics model further comprises: generating a plurality of candidate ligand atoms by selecting candidate ligand atoms from a plurality of non-hydrogen atoms of the ligand based on determining that atoms of the plurality of candidate ligand atoms are associated with a plurality of hydrogen bonds between the ligand and the protein, and generating a plurality of candidate protein atoms by selecting candidate protein atoms from the protein, wherein the candidate protein atoms are associated with the plurality of hydrogen bonds between the ligand and the protein.

CLAUSE 10. A non-transitory computer-readable medium storing instructions that, when executed by at least one processor, cause a computer system to: generate, based on simulated binding interactions between a ligand and a protein, a plurality of candidate atoms for a molecular dynamics model, generate a filtered set of candidate atoms by selecting a subset of candidate atoms from the plurality of candidate atoms based on angle metrics between the ligand and the protein for the subset of candidate atoms satisfying an angle metric threshold, generate positional restraints between the ligand and the protein by selecting, from the filtered set of candidate atoms, restraint atoms based on a loss function that combines a measure of angular variability that penalizes angular variability with an angular conditioning factor that penalizes collinear geometry, and generate a binding free energy between the ligand and the protein by applying the positional restraints during lambda windows of the molecular dynamics model.

CLAUSE 11. The non-transitory computer-readable medium of clause 10, further comprising instructions that, when executed by the at least one processor, cause the computer system to generate the positional restraints between the ligand and the protein by selecting the restraint atoms based on the loss function that combines the measure of angular variability and the angular conditioning factor with a measure of distance that penalizes distance between candidate atoms and a measure of distance variability that penalizes distance variability.

CLAUSE 12. The non-transitory computer-readable medium of any of clauses 10-11, further comprising instructions that, when executed by the at least one processor, cause the computer system to generate the positional restraints between the ligand and the protein by selecting the restraint atoms based on the loss function that combines a first measure of angular variability for a first angle for the restraint atoms and a second measure of angle variability for a second angle for the restraint atoms.

CLAUSE 13. The non-transitory computer-readable medium of any of clauses 10-12, further comprising instructions that, when executed by the at least one processor, cause the computer system to generate the positional restraints between the ligand and the protein by selecting the restraint atoms based on the loss function that combines a first measure of dihedral angular variability for the restraint atoms, a second measure of dihedral angular variability for the restraint atoms, and a third measure of dihedral angular variability for the restraint atoms.

CLAUSE 14. The non-transitory computer-readable medium of any of clauses 10-13, further comprising instructions that, when executed by the at least one processor, cause the computer system to apply an angular stabilization potential to an angle restraint of the positional restraints during the lambda windows of the molecular dynamics model, wherein the angular stabilization potential is configured to increase a restorative force as the angle restraint approaches a threshold angular geometry.

CLAUSE 15. The non-transitory computer-readable medium of any of clauses 10-14, further comprising instructions that, when executed by the at least one processor, cause the computer system to apply the positional restraints to the lambda windows of the molecular dynamics model by: applying the positional restraints during the lambda windows by applying dihedral restraints during a first set of lambda windows of the molecular dynamics model, and applying the positional restraints during the lambda windows by applying angle restraints during a second set of lambda windows of the molecular dynamics model.

CLAUSE 16. A system comprising: at least one processor, and at least one non-transitory computer-readable storage medium storing instructions that, when executed by the at least one processor, cause the system to: generate, based on simulated binding interactions between a ligand and a protein, a plurality of candidate atoms for a molecular dynamics model, generate a filtered set of candidate atoms by selecting a subset of candidate atoms from the plurality of candidate atoms based on angle metrics between the ligand and the protein for the subset of candidate atoms satisfying an angle metric threshold, generate positional restraints between the ligand and the protein by selecting, from the filtered set of candidate atoms, restraint atoms based on a loss function that combines a measure of angular variability that penalizes angular variability with an angular conditioning factor that penalizes collinear geometry, and generate a binding free energy between the ligand and the protein by applying the positional restraints during lambda windows of the molecular dynamics model.

CLAUSE 17. The system of any of clause 16, further comprising instructions that, when executed by the at least one processor, cause the system to generate the positional restraints between the ligand and the protein by selecting the restraint atoms based on the loss function that combines the measure of angular variability and the angular conditioning factor with a measure of distance that penalizes distance between candidate atoms and a measure of distance variability that penalizes distance variability.

CLAUSE 18. The system of any of clauses 16-17, further comprising instructions that, when executed by the at least one processor, cause the system to generate the positional restraints between the ligand and the protein by selecting the restraint atoms based on the loss function that combines a first measure of angular variability for a first angle for the restraint atoms and a second measure of angle variability for a second angle for the restraint atoms.

CLAUSE 19. The system of any of clauses 16-18, further comprising instructions that, when executed by the at least one processor, cause the system to generate the positional restraints between the ligand and the protein by selecting the restraint atoms based on the loss function that combines a first measure of dihedral angular variability for the restraint atoms, a second measure of dihedral angular variability for the restraint atoms, and a third measure of dihedral angular variability for the restraint atoms.

CLAUSE 20. The system of any of clauses 16-19, further comprising instructions that, when executed by the at least one processor, cause the system to optimize a set of lambda windows for annihilating a nonbonded interaction by: determining, utilizing a statistical free energy estimation algorithm, a first free energy estimation score for a candidate set of lambda windows, determining, utilizing the statistical free energy estimation algorithm, a second free energy estimation score for a modified candidate set of lambda windows, and selecting the set of lambda windows for annihilating the nonbonded interaction based on a variance between the first free energy estimation score and the second free energy estimation score.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., memory), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed by a processor, cause a general-purpose computer, special purpose computer, or special purpose processing device to perform a certain function or group of functions. In some embodiments, computer-executable instructions are executed by a general-purpose computer to turn the general-purpose computer into a special purpose computer implementing elements of the disclosure. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. As used herein, the term “cloud computing” refers to a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In addition, as used herein, the term “cloud-computing environment” refers to an environment in which cloud computing is employed.

FIG. 12 illustrates a block diagram of an example computing device 1200 that may be configured to perform one or more of the processes described above. One will appreciate that one or more computing devices, such as the computing device 1200 may represent the computing devices described above (e.g., server device(s) 1002, database 1008, client device(s) 1010, and third-party server device(s) 1014). In one or more embodiments, the computing device 1200 may be a mobile device (e.g., a mobile telephone, a smartphone, a PDA, a tablet, a laptop, a camera, a tracker, a watch, a wearable device, etc.). In some embodiments, the computing device 1200 may be a non-mobile device (e.g., a desktop computer or another type of client device). Further, the computing device 1200 may be a server device that includes cloud-based processing and storage capabilities.

As shown in FIG. 12, the computing device 1200 can include one or more processor(s) 1202, memory 1204, a storage device 1206, input/output interfaces 1208 (or “I/O interfaces 1208”), and a communication interface 1210, which may be communicatively coupled by way of a communication infrastructure (e.g., bus 1212). While the computing device 1200 is shown in FIG. 12, the components illustrated in FIG. 12 are not intended to be limiting. Additional or alternative components may be used in other embodiments. Furthermore, in certain embodiments, the computing device 1200 includes fewer components than those shown in FIG. 12. Components of the computing device 1200 shown in FIG. 12 will now be described in additional detail.

In particular embodiments, the processor(s) 1202 includes hardware for executing instructions, such as those making up a computer program. As an example, and not by way of limitation, to execute instructions, the processor(s) 1202 may retrieve (or fetch) the instructions from an internal register, an internal cache, memory 1204, or a storage device 1206 and decode and execute them.

The computing device 1200 includes memory 1204, which is coupled to the processor(s) 1202. The memory 1204 may be used for storing data, metadata, and programs for execution by the processor(s). The memory 1204 may include one or more of volatile and non-volatile memories, such as Random-Access Memory (“RAM”), Read-Only Memory (“ROM”), a solid-state disk (“SSD”), Flash, Phase Change Memory (“PCM”), or other types of data storage. The memory 1204 may be internal or distributed memory.

The computing device 1200 includes a storage device 1206 includes storage for storing data or instructions. As an example, and not by way of limitation, the storage device 1206 can include a non-transitory storage medium described above. The storage device 1206 may include a hard disk drive (HDD), flash memory, a Universal Serial Bus (USB) drive or a combination these or other storage devices.

As shown, the computing device 1200 includes one or more I/O interfaces 1208, which are provided to allow a user to provide input to (such as user strokes), receive output from, and otherwise transfer data to and from the computing device 1200. These I/O interfaces 1208 may include a mouse, keypad or a keyboard, a touch screen, camera, optical scanner, network interface, modem, other known I/O devices or a combination of such I/O interfaces 1208. The touch screen may be activated with a stylus or a finger.

The I/O interfaces 1208 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O interfaces 1208 are configured to provide graphical data to a display for presentation to a user. The graphical data may be representative of one or more graphical user interfaces and/or any other graphical content as may serve a particular implementation.

The computing device 1200 can further include a communication interface 1210. The communication interface 1210 can include hardware, software, or both. The communication interface 1210 provides one or more interfaces for communication (such as, for example, packet-based communication) between the computing device and one or more other computing devices or one or more networks. As an example, and not by way of limitation, communication interface 1210 may include a network interface controller (NIC) or network adapter for communicating with an Ethernet or other wire-based network or a wireless NIC (WNIC) or wireless adapter for communicating with a wireless network, such as a WI-FI. The computing device 1200 can further include a bus 1212. The bus 1212 can include hardware, software, or both that connects components of computing device 1200 to each other.

In the foregoing specification, the invention has been described with reference to specific example embodiments thereof. Various embodiments and aspects of the invention(s) are described with reference to details discussed herein, and the accompanying drawings illustrate the various embodiments. The description above and drawings are illustrative of the invention and are not to be construed as limiting the invention. Numerous specific details are described to provide a thorough understanding of various embodiments of the present invention.

The present invention may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

What is claimed is:

1. A computer-implemented method comprising:
generating, based on simulated binding interactions between a ligand and a protein, a plurality of candidate atoms for a molecular dynamics model;
generating a filtered set of candidate atoms by selecting a subset of candidate atoms from the plurality of candidate atoms based on angle metrics between the ligand and the protein for the subset of candidate atoms satisfying an angle metric threshold;
generating positional restraints between the ligand and the protein by selecting, from the filtered set of candidate atoms, restraint atoms based on a loss function that combines a measure of angular variability that penalizes angular variability with an angular conditioning factor that penalizes collinear geometry; and
generating a binding free energy between the ligand and the protein by applying the positional restraints during lambda windows of the molecular dynamics model.

2. The computer-implemented method of claim 1, further comprising generating the positional restraints between the ligand and the protein by selecting the restraint atoms based on the loss function that combines the measure of angular variability and the angular conditioning factor with a measure of distance that penalizes distance between candidate atoms and a measure of distance variability that penalizes distance variability.

3. The computer-implemented method of claim 1, further comprising generating the positional restraints between the ligand and the protein by selecting the restraint atoms based on the loss function that combines a first measure of angular variability for a first angle for the restraint atoms and a second measure of angle variability for a second angle for the restraint atoms.

4. The computer-implemented method of claim 1, further comprising generating the positional restraints between the ligand and the protein by selecting the restraint atoms based on the loss function that combines a first measure of dihedral angular variability for the restraint atoms, a second measure of dihedral angular variability for the restraint atoms, and a third measure of dihedral angular variability for the restraint atoms.

5. The computer-implemented method of claim 1, further comprising applying an angular stabilization potential to an angle restraint of the positional restraints during the lambda windows of the molecular dynamics model, wherein the angular stabilization potential is configured to increase a restorative force as the angle restraint approaches a threshold angular geometry.

6. The computer-implemented method of claim 1, further comprising applying the positional restraints to the lambda windows of the molecular dynamics model by:
applying the positional restraints during the lambda windows by applying dihedral restraints during a first set of lambda windows of the molecular dynamics model; and
applying the positional restraints during the lambda windows by applying angle restraints during a second set of lambda windows of the molecular dynamics model.

7. The computer-implemented method of claim 1, further comprising annihilating a first type of nonbonded interaction concurrently with applying dihedral restraints during the lambda windows of the molecular dynamics model.

8. The computer-implemented method of claim 1, further comprising optimizing a set of lambda windows for annihilating a type of nonbonded interaction by:
determining, utilizing a statistical free energy estimation algorithm, a first free energy estimation score for a candidate set of lambda windows;
determining, utilizing the statistical free energy estimation algorithm, a second free energy estimation score for a modified candidate set of lambda windows; and
selecting the set of lambda windows for annihilating the type of nonbonded interaction based on a variance between the first free energy estimation score and the second free energy estimation score.

9. The computer-implemented method of claim 1, wherein generating the plurality of candidate atoms for the molecular dynamics model further comprises:
generating a plurality of candidate ligand atoms by selecting candidate ligand atoms from a plurality of non-hydrogen atoms of the ligand based on determining that atoms of the plurality of candidate ligand atoms are associated with a plurality of hydrogen bonds between the ligand and the protein; and
generating a plurality of candidate protein atoms by selecting candidate protein atoms from the protein, wherein the candidate protein atoms are associated with the plurality of hydrogen bonds between the ligand and the protein.

10. A non-transitory computer-readable medium storing instructions that, when executed by at least one processor, cause a computer system to:
generate, based on simulated binding interactions between a ligand and a protein, a plurality of candidate atoms for a molecular dynamics model;
generate a filtered set of candidate atoms by selecting a subset of candidate atoms from the plurality of candidate atoms based on angle metrics between the ligand and the protein for the subset of candidate atoms satisfying an angle metric threshold;
generate positional restraints between the ligand and the protein by selecting, from the filtered set of candidate atoms, restraint atoms based on a loss function that combines a measure of angular variability that penalizes angular variability with an angular conditioning factor that penalizes collinear geometry; and
generate a binding free energy between the ligand and the protein by applying the positional restraints during lambda windows of the molecular dynamics model.

11. The non-transitory computer-readable medium of claim 10, further comprising instructions that, when executed by the at least one processor, cause the computer system to generate the positional restraints between the ligand and the protein by selecting the restraint atoms based on the loss function that combines the measure of angular variability and the angular conditioning factor with a measure of distance that penalizes distance between candidate atoms and a measure of distance variability that penalizes distance variability.

12. The non-transitory computer-readable medium of claim 10, further comprising instructions that, when executed by the at least one processor, cause the computer system to generate the positional restraints between the ligand and the protein by selecting the restraint atoms based on the loss function that combines a first measure of angular variability for a first angle for the restraint atoms and a second measure of angle variability for a second angle for the restraint atoms.

13. The non-transitory computer-readable medium of claim 10, further comprising instructions that, when executed by the at least one processor, cause the computer system to generate the positional restraints between the ligand and the protein by selecting the restraint atoms based on the loss function that combines a first measure of dihedral angular variability for the restraint atoms, a second measure of dihedral angular variability for the restraint atoms, and a third measure of dihedral angular variability for the restraint atoms.

14. The non-transitory computer-readable medium of claim 10, further comprising instructions that, when executed by the at least one processor, cause the computer system to apply an angular stabilization potential to an angle restraint of the positional restraints during the lambda windows of the molecular dynamics model, wherein the angular stabilization potential is configured to increase a restorative force as the angle restraint approaches a threshold angular geometry.

15. The non-transitory computer-readable medium of claim 10, further comprising instructions that, when executed by the at least one processor, cause the computer system to apply the positional restraints to the lambda windows of the molecular dynamics model by:
applying the positional restraints during the lambda windows by applying dihedral restraints during a first set of lambda windows of the molecular dynamics model; and
applying the positional restraints during the lambda windows by applying angle restraints during a second set of lambda windows of the molecular dynamics model.

16. A system comprising:
at least one processor; and
at least one non-transitory computer-readable storage medium storing instructions that, when executed by the at least one processor, cause the system to:
generate, based on simulated binding interactions between a ligand and a protein, a plurality of candidate atoms for a molecular dynamics model;
generate a filtered set of candidate atoms by selecting a subset of candidate atoms from the plurality of candidate atoms based on angle metrics between the ligand and the protein for the subset of candidate atoms satisfying an angle metric threshold;
generate positional restraints between the ligand and the protein by selecting, from the filtered set of candidate atoms, restraint atoms based on a loss function that combines a measure of angular variability that penalizes angular variability with an angular conditioning factor that penalizes collinear geometry; and
generate a binding free energy between the ligand and the protein by applying the positional restraints during lambda windows of the molecular dynamics model.

17. The system of claim 16, further comprising instructions that, when executed by the at least one processor, cause the system to generate the positional restraints between the ligand and the protein by selecting the restraint atoms based on the loss function that combines the measure of angular variability and the angular conditioning factor with a measure of distance that penalizes distance between candidate atoms and a measure of distance variability that penalizes distance variability.

18. The system of claim 16, further comprising instructions that, when executed by the at least one processor, cause the system to generate the positional restraints between the ligand and the protein by selecting the restraint atoms based on the loss function that combines a first measure of angular variability for a first angle for the restraint atoms and a second measure of angle variability for a second angle for the restraint atoms.

19. The system of claim 16, further comprising instructions that, when executed by the at least one processor, cause the system to generate the positional restraints between the ligand and the protein by selecting the restraint atoms based on the loss function that combines a first measure of dihedral angular variability for the restraint atoms, a second measure of dihedral angular variability for the restraint atoms, and a third measure of dihedral angular variability for the restraint atoms.

20. The system of claim 16, further comprising instructions that, when executed by the at least one processor, cause the system to optimize a set of lambda windows for annihilating a nonbonded interaction by:
determining, utilizing a statistical free energy estimation algorithm, a first free energy estimation score for a candidate set of lambda windows;
determining, utilizing the statistical free energy estimation algorithm, a second free energy estimation score for a modified candidate set of lambda windows; and
selecting the set of lambda windows for annihilating the nonbonded interaction based on a variance between the first free energy estimation score and the second free energy estimation score.
